(19) United States
(12) Patent Application Publication
(10) Pub. No.: US 2021/0123077 A1
(43) Pub. Date: Apr. 29, 2021

(54) ADENO-ASSOCIATED VIRUS VECTOR MEDIATED GENE THERAPY FOR OPHTHALMIC DISEASES

(71) Applicant: Ocugen, Inc., Malvern, PA (US)

(72) Inventors: Rasappa Arumugham, Lansdale, PA (US); Arun Upadhyay, West Chester, PA (US); Neena B. Haider, Brookline, MA (US)

(73) Assignee: Ocugen, Inc., Malvern, PA (US)

(21) Appl. No.: 17/080,078

(22) Filed: Oct. 26, 2020

Related U.S. Application Data
(63) Continuation-in-part of application No. PCT/US19/46904, filed on Aug. 16, 2019.
(60) Provisional application No. 62/839,672, filed on Apr. 27, 2019.

(51) Int. Cl. C12N 15/86; C12N 7/00; C07K 14/705; A61P 27/02; A61K 9/00

(52) U.S. Cl. CPC C12N 15/86; C12N 7/00; C07K 14/70564; A61P 27/02; A61K 48/00; C12N 2830/50; C12N 2750/14143; C12N 2750/14171; A61K 9/0051

(57) ABSTRACT

The present invention provides compositions and methods for treating an ocular condition and/or disease. In particular, compositions and methods of the invention are directed to a gene therapy for treatment of an ocular condition and/or disease. One particular aspect of the invention provides a recombinant DNA comprising (i) a therapeutic gene, a functional counterpart of a defective gene associated with manifestation said ocular condition or disease, or a combination thereof; and (ii) a delivery vehicle adapted for delivering said gene of (i) to cells in target ocular area for treating said ocular condition or disease, said delivery vehicle comprising an adeno-associated virus (AAV) serotype.

### CROSS-REFERENCE TO RELATED APPLICATIONS

This application is continuation-in-part application of PCT patent application number PCT/US19/46904, filed Aug. 16, 2019, which claims the priority benefit of U.S. Provisional Application No. 62/839,672, filed Apr. 27, 2019, all of which are incorporated herein by reference in its entirety.

### FIELD OF THE INVENTION

The present invention relates to a composition and a method for gene therapy in treating an ocular condition and/or disease. In particular, the present invention relates to a recombinant DNA that comprises (i) a gene selected from the group consisting of: (a) a therapeutic gene capable of ameliorating the ocular condition or disease in said subject, (b) a functional counterpart of a defective gene associated with manifestation said ocular condition or disease, and (c) a combination thereof; and (ii) a delivery vehicle adapted for delivering said gene to cells in an ocular area for treating said ocular condition or disease, wherein said delivery vehicle comprises an adeno-associated virus (AAV) serotype. The present invention also relates to a plasmid comprising the same and a recombinant adeno-associated virus (rAAV) vector comprising: a gene selected from the group consisting of: (a) a therapeutic gene capable of ameliorating the ocular condition or disease in said subject, (b) a functional counterpart of a defective gene associated with manifestation said ocular condition or disease, and (c) a combination thereof.

### BACKGROUND OF THE INVENTION

Adeno-associated virus (AAV) have great relevance as gene transfer vectors. In fact, adeno-associated virus vectors are currently among the most frequently used viral vectors for gene therapy. Until recently, AAV has not been of great interest due to a lack of widespread knowledge of the virus. In particular, because AAV is non-pathogenic, it has not been studied widely in medical field. To date twelve human serotypes of AAV (AAV serotype 1 [AAV-1] to AAV-12) and more than 100 serotypes from nonhuman primates have been identified. Interestingly, this lack of pathogenicity of the virus makes it an ideal candidate as a delivery vehicle for gene therapy applications.

Using gene therapy to treat various clinical conditions have gained a tremendous interest. While the majority of gene therapies are targeted to correcting a clinical condition brought on by a defective gene of only a limited diseases, such as sickle cell anemia and cystic fibrosis, other non-genetically induced clinical conditions (e.g., cancer) are also being tested with a gene therapy.

Recently, Luxturna™, a gene therapy product received FDA approval for the treatment for RPE65 mutation associated Leber congenital amaurosis (LCA), LCA-2. This mutation represents one of more than 250 gene mutations implicated in retinal degeneration identified so far. Still, no gene therapy have been approved for ocular conditions or ocular diseases such as other types of LCA, retinitis pigmentosa, enhance S-cone syndrome, Goldmann Favre syndrome, rod-cone dystrophy, Bardet-Biedl Syndrome, Achromatopsia, Best Disease (vitelliform macular degeneration), Bardet-Biedl Syndrome, Choroideremia, Macular Degeneration, Stargardt Disease, X-Linked Retinoschisis (XLRS), X-Linked Retinitis Pigmentosa (XLRP), Usher Syndrome, cone-rod dystrophy, Dry-Age related macular degeneration, wet-Age related macular degeneration, etc.

Even traditional pharmaceutical methods are not available for treating many of these ocular conditions or diseases, and they require a development of novel therapeutics to address this unmet medical need. Therefore, there is a need for a new therapeutic methods and compositions for treating various ocular diseases and/or conditions associated with retinal degeneration.

### SUMMARY OF THE INVENTION

The present invention provides compositions and methods for treating a various ocular diseases or conditions using a gene therapy. One particular aspect of the invention provides a recombinant DNA comprising:
(i) a gene selected from the group consisting of: (a) a therapeutic gene, (b) a functional counterpart of a defective gene associated with manifestation an ocular condition or disease, and (c) a combination thereof; and
(ii) a delivery vehicle adapted for delivering said gene of (i) to cells in target ocular area for treating an ocular condition or disease, said delivery vehicle comprising an adeno-associated virus (AAV) serotype.

In some embodiments, the gene (i) is packaged or encapsulated within said delivery vehicle. Still in other embodiments, the delivery vehicle includes an inverted terminal repeat (ITR) of AAV (“AAV ITR”). Yet in other embodiments, the AAV ITR is AAV2 ITR.

Still in other embodiments, the recombinant DNA further comprises (i) AAV2 ITR, (ii) a promotor, (iii) an enhancer, (iv) a polyadenylation moiety, (v) a regulatory switch to control expression, or (vi) a combination thereof.

Yet in other embodiments, the polyadenylation moiety comprises simian virus 40 (SV40) polyadenylation (PolyA) region, bovine growth hormone (bGH) PolyA region, or a combination thereof.

In other embodiments, the therapeutic gene is selected from the group consisting of:
(a) human nuclear hormone receptor (hNHR) gene or a fragment thereof, wherein said hNHR gene is selected from the group consisting of NR2E3, NR1C3, NR1D1, RORA, NUPR1, NR2C1, and LXRa;
(b) a growth factor or an angiogenic modulator gene that encodes a protein selected from the group consisting of:
   (i) anti-vegf:
   (ii) lens epithelium derived growth factor;
   (iii) tumstatin;
   (iv) transferrin and tumstatin fusion protein;
   (v) fibroblast growth factor;
   (vi) platelet-derived growth factor family;
   (vii) vascular endothelial growth factor sub-family;
   (viii) epidermal growth factor family;
   (ix) fibroblast growth factor family;
   (x) transforming growth factor-β superfamily (e.g., TGF-β1; activins; follistatin and bone morphogenetic proteins);
   (xi) angiopoietin-like family;
   (xii) galectins family;
   (xiii) integrin superfamily, as well as pigment epithelium derived factor;
   (xiv) hepatocyte growth factor;
   (xv) angiopoietins;
   (xvi) endothelins;
   (xvii) hypoxia-inducible factors;
   (xviii) insulin-like growth factors;
   (xix) cytokines; and
   (xx) matrix metalloproteinases gene or a fragment thereof; and
(c) a combination thereof.

Yet still in other embodiments, the functional counterpart of a defective gene comprises a gene associated with retinal degeneration such as LCA (e.g., CRX, AIPL1, TULP1, CABP4, RPE65, CEP290, and other genes known to one skilled in the art); RP (e.g., CRX, NRL, NR2E3, PRPH2, RHO, ROM1, RPE65, ABCA4, MERTK, NRL, PDE6A, PDE6B, SAG, TULP1 and other genes known to one skilled in the art); Cone-rod dystrophy (e.g., AIPL1, CRX, PRPH2, ABCA4, CNGB3, RAB28, CACNA1F, RPGR, and other genes known to one skilled in the art); Macular degeneration (e.g., PRPH2, ELOV4, ANCA4, RPGR and other genes known to one skilled in the art); congenital stationary night blindness (e.g., GNAT1, PDE6B, RHO, CABP4, GRK1, SAG, CANA1F, and other genes known to one skilled in the art); synaptic diseases (e.g., CACNA2D4, CACNA1F, XLRS, and other genes known to one skilled in the art); Bardet-Biedl syndrome (e.g., BBS2, BBS4, BBS6, CEP290, and other genes known to one skilled in the art); Joubert syndrome (e.g., CEP290 and other genes known to one skilled in the art); Senior-Loken syndrome (e.g., CEP290 and other genes known to one skilled in the art); and Usher syndrome (e.g., MYO7A, USH2A, and other genes known to one skilled in the art).

Yet still in other embodiments, the therapeutic gene, and the functional counterpart of the disease associated defective gene can be administered to patients individually either at same time or at different time points one after the other in any sequence; or in combinations at the same time; or in single or multiple administrations.

Some of the genes that can be used to produce vectors and recombinant DNA of the invention are shown in odd numbered sequences in SEQ ID NOs: 1-69. It should be appreciated that the gene sequence (e.g., the oligonucleotide sequence) in odd numbered sequences in SEQ ID NOs: 1-69 can vary as long as it produces the corresponding protein sequence provided in even numbered sequences shown in SEQ ID NOs: 2-70 or at least the active portion of even numbered sequences shown in SEQ ID NOs: 2-70.

In some embodiments, genes can encode full-length or fragment of an identified protein thereof. Yet in other embodiments, these genes have at least about 90%, often at least about 95%, sequence identity to full-length wild-type counterpart gene across full or function regions of the genes and show associated activity. The sequence of useful genes of the present invention are readily available to one skilled in the art, such as in the gene databank at national center for biotechnology information (NCBI).

Still in other embodiments, the recombinant DNA further comprises (i) a promotor, (ii) an enhancer, (iii) a polyadenylation moiety, or (iv) a combination thereof. In some instances, the polyadenylation moiety comprises simian virus 40 (SV40) polyadenylation (PolyA) region, bovine growth hormone (bGH) PolyA region, or a combination thereof.

Yet in other embodiments, the recombinant DNA further comprises cytomegalovirus (CMV) promoter or enhancer, elongation factor 1a (EF1a), chicken β-actin (CBA) promoter, a CAG promotor, a cell/tissue specific promoter (such as Rho, RK, opsin promoter, or others known to one skilled in the art), or a combination thereof.

Another aspect of the invention provides a plasmid comprising a recombinant DNA described herein.

Still another aspect of the invention provides a recombinant adeno-associated virus (rAAV) vector comprising:
(i) a therapeutic gene, wherein said therapeutic gene is selected from the group consisting of:
   (a) human nuclear hormone receptor (hNHR) gene or a fragment thereof, wherein said hNHR gene is selected from the group consisting of NR2E3, NR1C3, NR1D1, RORA, NUPR1, NR2C1, and LXRa;
   (b) a growth factor and/or an angiogenic modulator such anti-vegf, lens epithelium derived growth factor, tumstatin; fusion of transferrin and tumstatin protein; fibroblast growth factor; platelet-derived growth factor family; vascular endothelial growth factor sub-family; epidermal growth factor family; fibroblast growth factor family; transforming growth factor-β superfamily (e.g., TGF-β1, activins, follistatin and bone morphogenetic proteins); angiopoietin-like family; galectins family; integrin superfamily, as well as pigment epithelium derived factor; hepatocyte growth factor; angiopoietins; endothelins; hypoxia-inducible factors; insulin-like growth factors; cytokines; and matrix metalloproteinases gene or a fragment thereof; and
   (c) a combination thereof; and
(ii) a functional counterpart of a defective gene associated with manifestation an ocular condition or disease such as LCA (e.g., CRX, AIPL1, TULP1, CABP4, RPE65, CEP290, and others); RP (e.g., CRX, NRL, NR2E3, PRPH2, RHO, ROM1, RPE65, ABCA4, MERTK, NRL, PDE6A, PDE6B, SAG, TULP1 and others); Cone-rod dystrophy (e.g., AIPL1, CRX, PRPH2, ABCA4, CNGB3, RAB28, CACNA1F, RPGR, and others); Macular degeneration (e.g., PRPH2, ELOV4, ANCA4, RPGR and others); congenital stationary night blindness (e.g., GNAT1, PDE6B, RHO, CABP4, GRK1, SAG, CANA1F, and others); synaptic diseases (e.g., CACNA2D4, CACNA1F, XLRS, and others); Bardet-Biedl syndrome (e.g., BBS2, BBS4, BBS6, CEP290, and others); Joubert syndrome (e.g., CEP290); Senior-Loken syndrome (e.g., CEP290); Usher syndrome (e.g., MYO7A, USH2A, and others); or
(iii) a combination thereof.

In some embodiments, the rAAV vector further comprises a naturally occurring (i.e., wild-type or “normal functioning type”) gene that encodes adeno-associated virus (AAV) serotype capsid protein. In some instances, the naturally occurring AAV serotype is selected from the group consisting of AAV1 (SEQ ID NO: 71), AAV2 (SEQ ID NO: 72), AAV5 (SEQ ID NO: 73), and AAV8 (SEQ ID NO: 74).

In one particular embodiment, the NHR gene is selected from the group consisting of Nr2e3, Nr1d1, Rora, Nupr1, Nr2c1, and LXR. In some instances, the Nr2e3 gene encodes full-length Nr2e3 protein or a fragment thereof. In one particular embodiment, the Nr2e3 gene comprises SEQ ID NO:1. Yet in another embodiment, the Nr2e3 gene has at least about 80%, typically, at least about 85%, often at least about 90%, and most often at least 95% sequence identity to SEQ ID NO:1.

Yet in some embodiments, the Nr1d1 gene encodes full-length Nr1d1 protein or a fragment thereof. Still in another embodiment, the Nr1d1 gene comprises SEQ ID NO:5. In some instances, the Nr1d1 gene has at least about 80%, typically, at least about 85%, often at least about 90%, and most often at least 95% sequence identity to SEQ ID NO:5.

Still in some embodiments, the RORA gene encodes full-length RORA protein or fragment of thereof. In one particular embodiment, the RORA gene comprises SEQ ID NO:7. In some instances, the RORA gene has at least about 80%, typically, at least about 85%, often at least about 90%, and most often at least 95% sequence identity to SEQ ID NO:7.

In other embodiments, the NR1C3 gene encodes full-length NR1C3 protein or a fragment thereof. In some embodiments, the NR1C3 gene comprises SEQ ID NO:3. Still in other embodiments, the NR1C3 gene has at least about 80%, typically, at least about 85%, often at least about 90%, and most often at least 95% sequence identity to SEQ ID NO:3.

In further embodiments, the NR2C1 gene encodes full-length NR2C1 protein or fragment of thereof. Still in another embodiment, the NR2C1 gene comprises SEQ ID NO:11. Yet in other embodiments, the NR2C1 gene has at least about 80%, typically, at least about 85%, often at least about 90%, and most often at least 95% sequence identity to SEQ ID NO:11.

Still in other embodiments, the NUPR1 gene encodes full-length NUPR1 protein or fragment of thereof. Yet in other embodiments, the NUPR1 gene comprises SEQ ID NO:9. Further, in other embodiments, the NUPR1 gene has at least about 80%, typically, at least about 85%, often at least about 90%, and most often at least 95% sequence identity to SEQ ID NO:9.

In other embodiments, the LXRa gene encodes full-length LXRa protein or fragment of thereof. In some instances, the LXRa gene comprises SEQ ID NO:13. Still in other instances, the LXRa gene has at least about 80%, typically, at least about 85%, often at least about 90%, and most often at least 95% sequence identity to SEQ ID NO:13.

Yet in other embodiments, the rAAV comprises full or functional copy of a diseases defective gene described herein and exemplified in odd numbered sequences in SEQ ID NOs:1-69, in particular odd numbered sequences in SEQ ID NO:15-69. In some embodiments, these genes encode full-length or fragment of an identified protein thereof. Yet in other embodiments, these genes have at least about 80%, typically, at least about 85%, often at least about 90%, and most often at least 95% sequence identity to full-length wild-type counterpart gene.

Yet in other embodiments, the rAAV vector further comprises a gene that encodes capsid protein having SEQ ID NO:71, 72, 73, or 74.

In one particular embodiment, the NHR gene is a human NHR (hNHR) gene.

Another aspect of the invention provides a pharmaceutical composition comprising a recombinant adeno-associated virus (rAAV) vector disclosed herein.

Another aspect of the invention provides a method for treating an ocular condition or ocular disease, said method comprising administering to an ocular tissue of a subject in need of such a treatment a therapeutically effective amount of a composition comprising a recombinant adeno-associated virus (rAAV) vector disclosed herein to treat said subject, wherein said ocular tissue is selected from the group consisting of retinal tissue, choroid tissue, and vitreous tissue.

In some embodiments, the composition comprising rAAV vector is suitably dispersed in a pharmacologically acceptable formulation.

Yet in other embodiments, the administration occurs more than once.

Still in other embodiments, the amount of viral particles administered to the subject ranges from about 10⁵ to about 10²⁰, typically from about 10⁶ to about 10¹⁹, often from about 10⁷ to about 10¹⁵, and more often from about 10⁸ to about 10¹⁴.

In other embodiments, the ocular condition or ocular disease comprises Leber congenital amaurosis (LCA), retinitis pigmentosa, enhance S-cone syndrome, Goldmann Favre syndrome, rod-cone dystrophy Bardet-Biedl Syndrome, Achromatopsia, Best Disease (vitelliform macular degeneration), Bardet-Biedl Syndrome, Choroideremia, Macular Degeneration, Stargardt Disease, X-Linked Retinoschisis (XLRS), X-Linked Retinitis Pigmentosa (XLRP), Usher Syndrome, cone-rod dystrophy, Dry-Age related macular degeneration, wet-Age related macular degeneration, or a combination thereof.

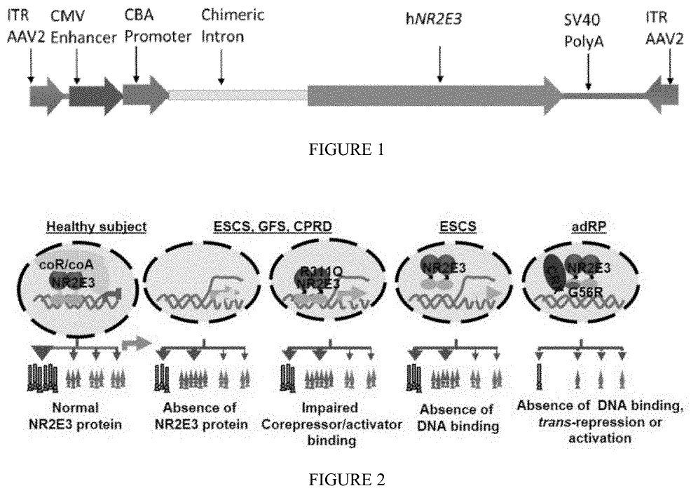

Yet another aspect of the invention provides a recombinant DNA or vector comprising (1) an oligonucleotide having at least about 90%, typically at least about 95%, often at least about 98%, more often at least about 99%, and most often 100% sequence identity to SEQ ID NO: 71, 72, 73, or 74 in combination with (2) an oligonucleotide having at least 90% sequence identity, typically at least about 95%, often at least about 98%, more often at least about 99%, and most often 100% sequence identity to any one of odd numbered sequence in SEQ ID NOs: 1-69. It should be appreciated that the scope of invention includes any combination of oligonucleotide of (i) and (ii). Exemplary combinations of oligonucleotides (i) and (ii) include, but are not limited to, SEQ ID NO:71 with any odd numbered sequence of SED ID NOs: 1-69 (e.g., SEQ ID NO: 1, 3, 5, 7, . . . 69, etc.), SEQ ID NO:72 with any odd numbered sequence of SED ID NOs: 1-69 (e.g., SEQ ID NO: 1, 3, 5, 7, . . . 69, etc.), SEQ ID NO:73 with any odd numbered sequence of SED ID NOs: 1-69 (e.g., SEQ ID NO: 1, 3, 5, 7, . . . 69, etc.), and SEQ ID NO:74 with any odd numbered sequence of SED ID NOs: 1-69 (e.g., SEQ ID NO: 1, 3, 5, 7, . . . 69, etc.). In some embodiments, the recombinant DNA can include more than one oligonucleotide of (ii), i.e., more than one of odd sequence number in SEQ ID NO:1-69. It should be appreciated that the terms “one of odd sequence number in SEQ ID NO:1-69” and “one of odd numbered sequence in SEQ ID NOs:1-69” are used interchangeably herein and mean SEQ ID NO:1, 3, 5, 7, 9, 11, 13, 15, 17, . . . 61, 63, 65, 67, or 69. Similarly, the terms “one of even sequence number in SEQ ID NOs:2-70” and “one of even numbered sequence in SEQ ID:2-70” are used interchangeably herein and mean SEQ ID NO:2, 4, 6, 8, 10, 12, . . . 60, 62, 64, 66, 68, or 70. In some embodiments, the recombinant DNA or vector also includes (a) a promotor, (b) an enhancer, (c) a polyadenylation moiety, or (d) a combination thereof. In some embodiments, the recombinant DNA or vector comprises those illustrated in FIG. 1, where AAV portion can be any one of SEQ ID NOs:71-74 and h/NR2E3 can be replaced with any one of odd numbered sequence in SEQ ID NO:1-69. In this manner, a wide variety of combination of recombinant DNAs and vectors are encompassed within the scope of the invention.

### BRIEF DESCRIPTION OF THE INVENTION

FIG. 1 is a schematic illustration of one particular adeno-associated virus serotype 5-based vector comprising the human NR2E3 gene expression cassette containing: a) AAV2 ITR; b) the cytomegalovirus (CMV) enhancer; c) the chicken beta actin (CBA) promoter; d) chimeric intron; e) the cloned cDNA coding for human NR2E3 protein (44692 dalton); and f) the SV40 polyadenylation (PolyA) region.

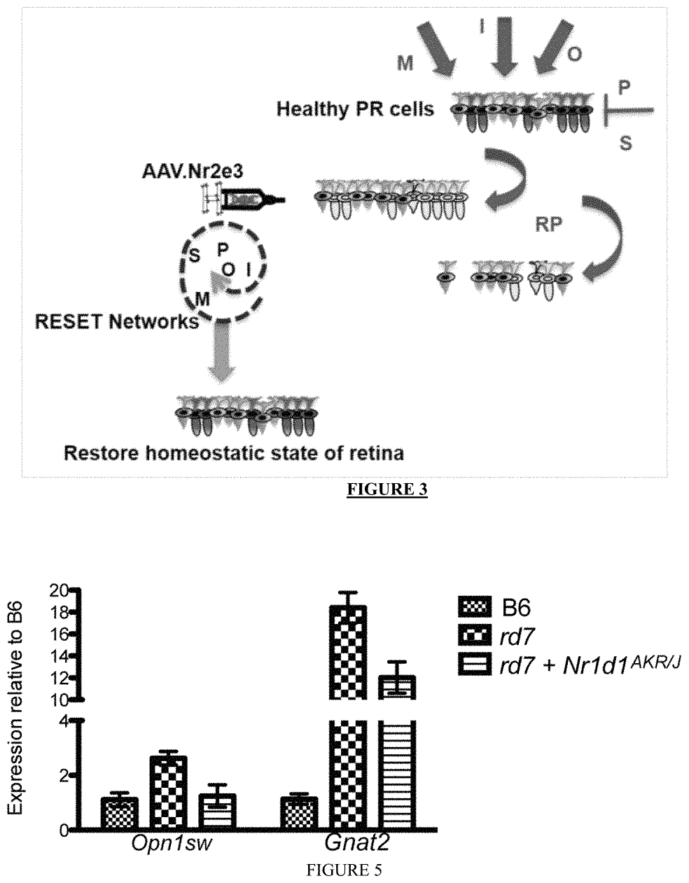

FIG. 2 is a schematic representation of the potential mechanism impacting Nr2e3 retinal degeneration, where coR/coA is corepressor or coactivator; ESCS is Enhanced S-cone syndrome; GFS is Goldman Favre syndrome; adRP is autosomal dominant retinitis pigmentosa; rod photoreceptors in grey and cone photoreceptors are in blue, green, and red.

FIG. 3 is a Schematic representation of potential Nr2e3 mediated therapy. Nr2e3 potentially resets key gene networks that contribute to retinal degeneration in RP. RP—retinitis pigmentosa; PR—photoreceptor cells; Gene networks: M—Metabolism; I—Inflammation; O—oxidative stress; P—photoreceptor genes; S—cell survival.

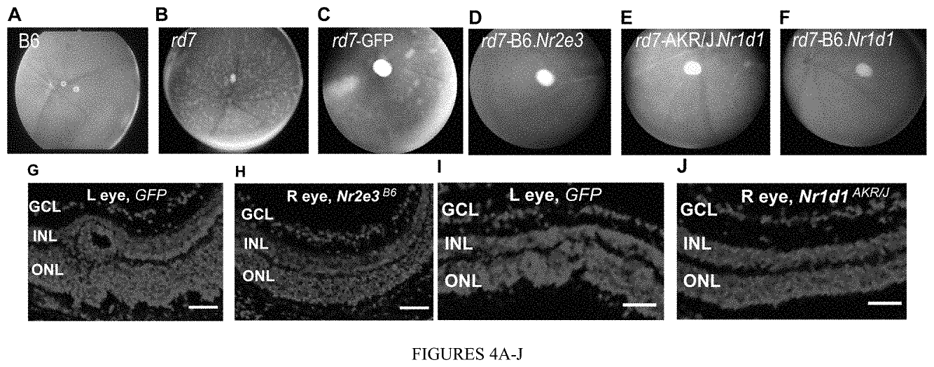

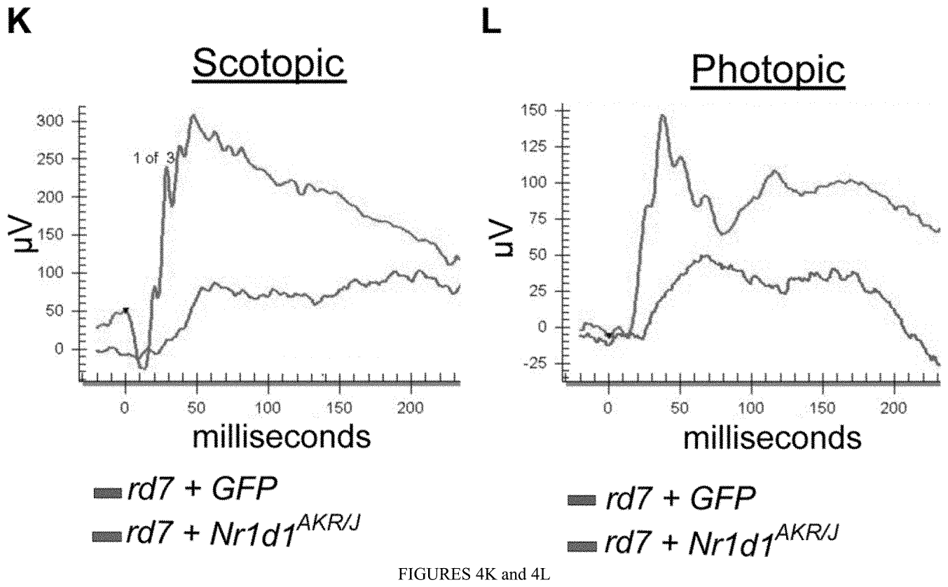

FIG. 4A is Fundus photograph of control B6 (uninjected) mouse retina.

FIG. 4B is Fundus photograph of control rd7 (uninjected) mouse retina.

FIG. 4C is Fundus photograph of GFP injected mouse retina.

FIG. 4D is Fundus photograph of GFP.Nr2e3B6 injected mouse retina.

FIG. 4E is Fundus photograph of GFP.Nr1d1AKR/J injected mouse retina.

FIG. 4F is Fundus photograph of GFP.Nr1d1B6 injected mouse retina.

FIGS. 4G-4J are DAPI staining (blue) showing rescue of defects in retinal morphology 30 days after electroporation into rd7 neonatal retinas, where L: left, R: right, GCL: ganglion cell layer, INL: inner nuclear layer, ONL: outer nuclear layer. Scale bar=50 mm. FIG. 4G is GFP control; FIG. 4H is Nr2e3B6 injected; FIG. 4I is GFP control; FIG. 4J is Nr1d1AKR/J injected.

FIG. 4K is a representative scotopic electroretinogram from animals 4 month after injection with GFP (blue) or GFP.Nr1d1AKR/J (red).

FIG. 4L is a representative photopic electroretinogram from animals 4 month after injection with GFP (blue) or GFP.Nr1d1AKR/J (red).

FIG. 5 is a graph showing expression of phototransduction genes Opn1sw and Gnat2 is rescued in rd7 retinas upon Nr1d1 delivery. Quantitative real time PCR (Polymerase chain reaction) shows that Nr1d1 delivery results in down-regulation of the phototransduction genes Opn1sw and Gnat2 in rd7 retinas (mean±SD of mean, n=3, p<0.05), to near normal level in a preclinical model.

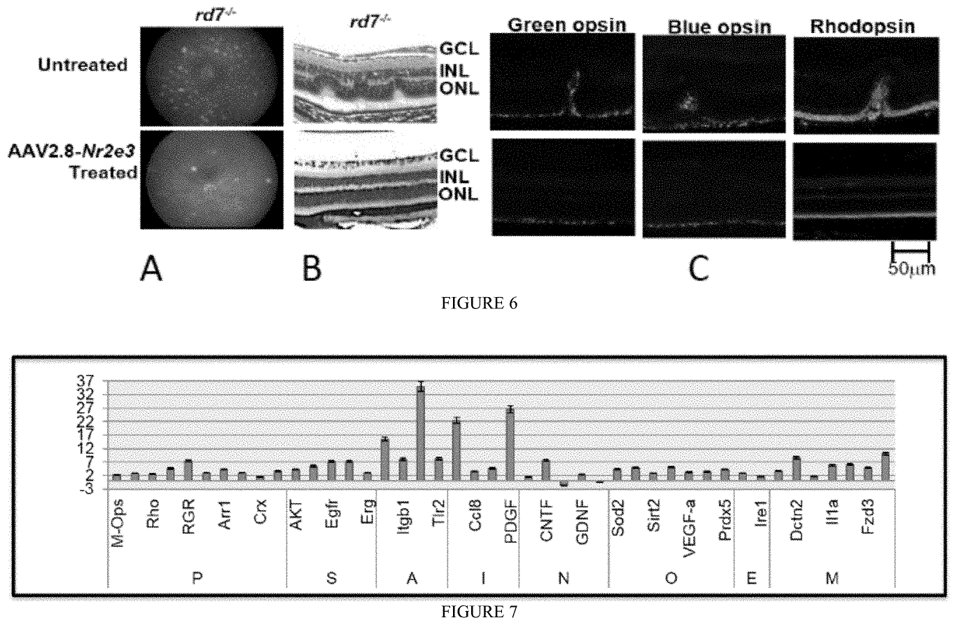

FIG. 6 is photos showing AAV2.8-mNr2e3 neonatal delivery prevents rd7 associated retinal degeneration. A. fundus of rd7; B. histology, hematoxylin/eosin staining C. immunohistochemistry cone (green and blue opsin) and rod (rhodopsin) expression shows prevention of degeneration following AAV-Nr2e3. Animals were injected at postnatal (P)0 and evaluated at 3 months. N>5.

FIG. 7 is a graph showing AAV2.8-mNr2e3 treated rd7 retinas exhibit a reset of homeostatic state in over 40 genes in seven gene networks. Real time PCR evaluation of approximately 75 genes belonging to seven different Nr2e3-regulated gene networks show over 40 genes are differentially regulated in treated vs untreated retinas. Figure shows those genes that had a fold variance change equal to or higher than 1.5. Networks: P. Phototransduction, S: Cell Survival, A: Apoptosis, I: Immunity/inflammation, N: Neuroprotection, O: Oxidative Stress, E: ER stress, M: Metabolism.

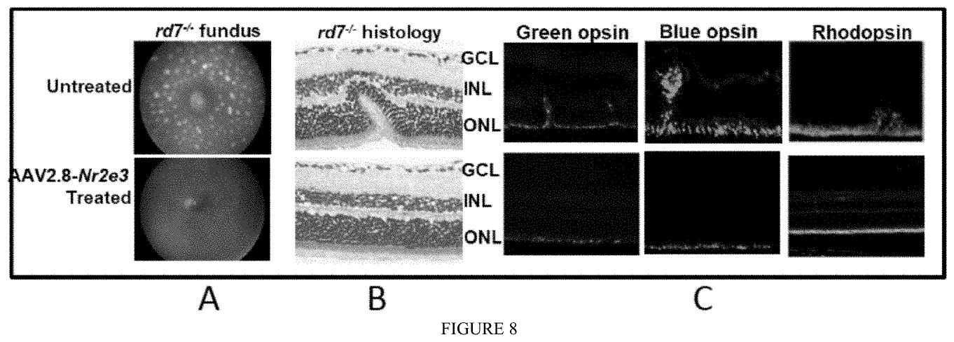

FIG. 8 is photos showing AAV2.8-mNr2e3 delivery at early to intermediate stage of; reverses rd7 associated retinal degeneration. A. fundus of rd7; B. histology, hematoxylin/eosin staining C. immunohistochemistry cone (green and blue opsin) and rod (rhodopsin) expression shows reversal of degeneration following subretinal AAV2.8-Nr2e3 delivery. Animals were injected at P21 and evaluated at 3 months. N>5.

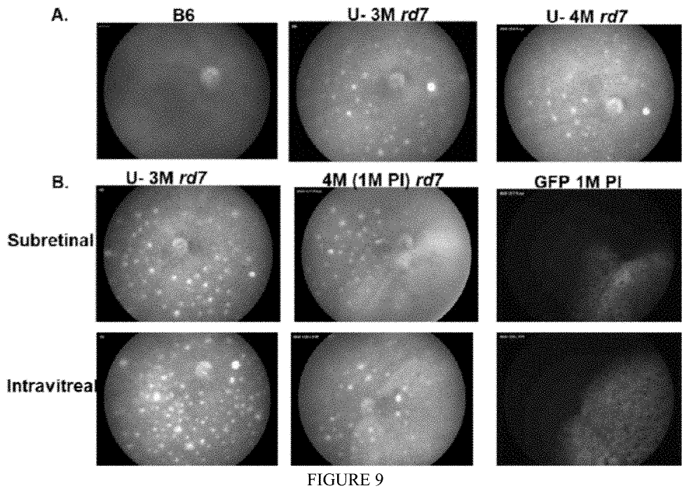

FIG. 9 is photos showing AAV5-mNr2e3 rescue of rd7 clinical phenotype. Panel A shows normal (B6), and rd7 and 3 and 4 months (M). Panel B shows 3M uninjected rd7 retina and the same retina, 1M post injection with subretinal and intravitreal routes of administration. Presence of GFP is observed indicating delivery of AAV5-Nr2e3 to the retina.

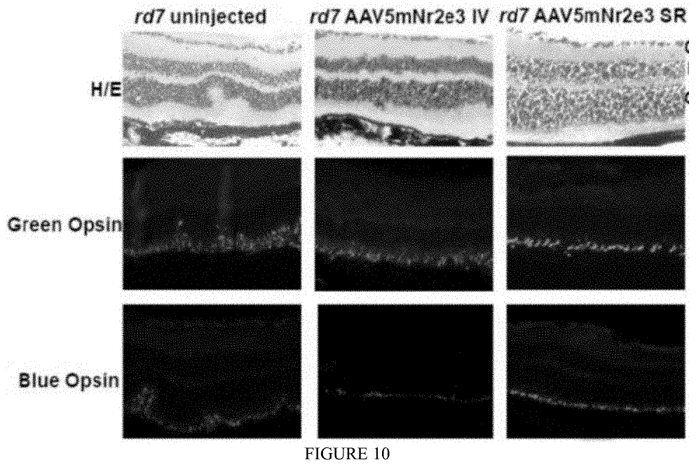

FIG. 10 is photos showing AAV5-mNr2e3 rescue of rd7 morphology. Hematoxylin and eosin (H&E) stain and blue and green opsin expression show resolution of whorls and photoreceptor cells in rd7 mice. Intravitreal (IV), subretinal (SR), ganglion cell layer (GCL), inner nuclear layer (INL), and outer nuclear layer (ONL).

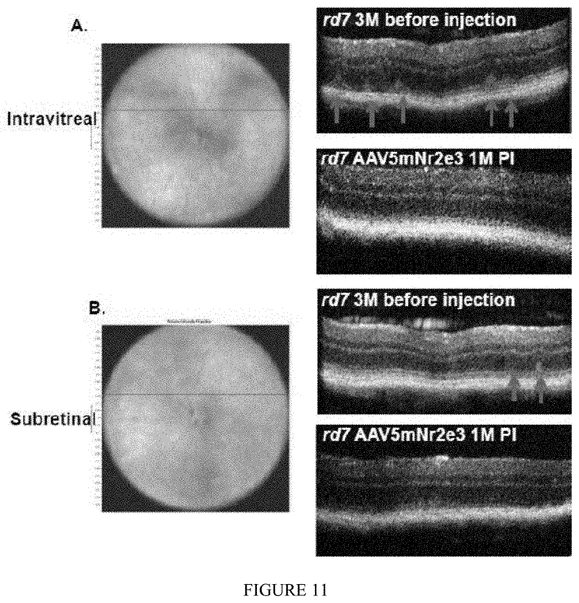

FIG. 11 is photos showing AAV-mNr2e3 rescue of rd7 clinical and histological phenotype. Optical coherence tomography (OCT) of A. intravitreal and B. subretinal injections of AAV5-Nr2e3 in rd7 mice. Whole retina image shows frame location of each scan denoted by green line. Right panel of scans taken at the same frame before and 1M after AAV5-Nr2e3 injection. Red arrow indicates whorls present in the scan before injection and resolved in scan 1M post injection (PI).

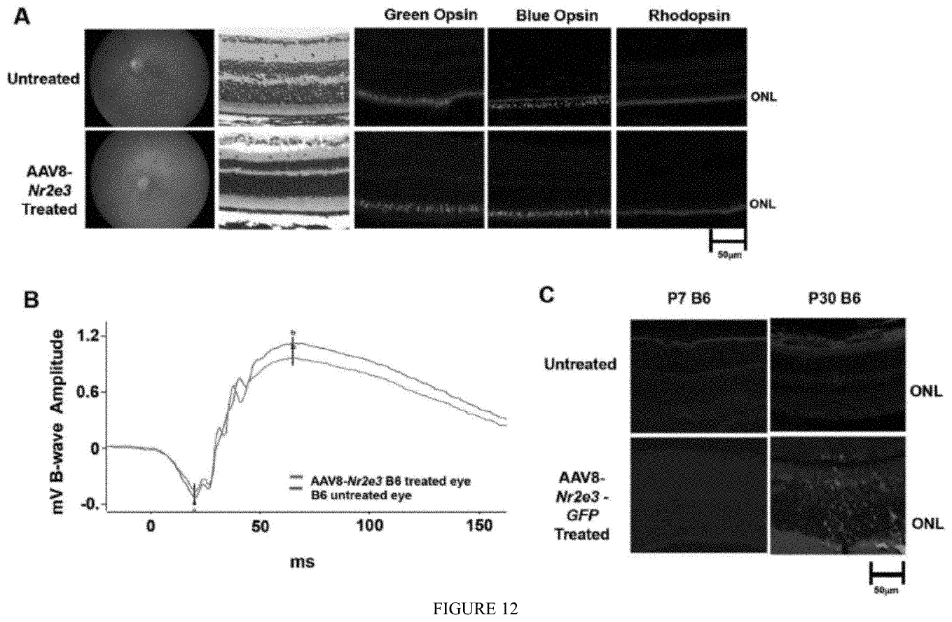

FIG. 12 is a collection of data showing overexpression of AAV8-Nr2e3 has no detrimental effects on the retina. Panel (A) is fundus, hematoxylin/eosin histology staining, and blue, green, and rhodopsin labeling of photoreceptor cells of B6 control AAV8-Nr2e3 treated animals; Panel (B) shows ERG response of control B6 treated and untreated. Animals injected at P0, tissue collected at P30. Panel (C) shows GFP label of AAV8-Nr2e3-GFP injected at P0, GFP expression assessed at P7 and P30. N=5.

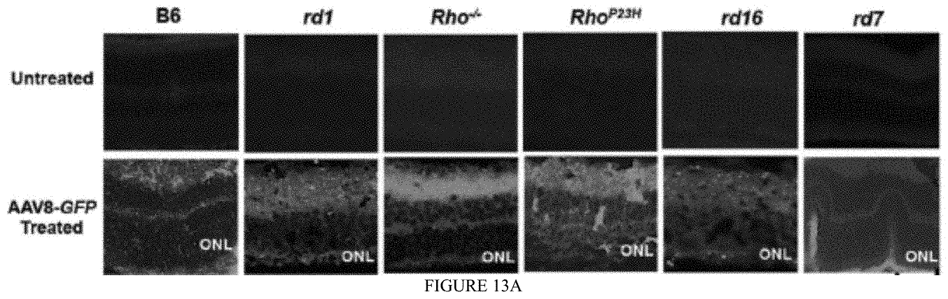

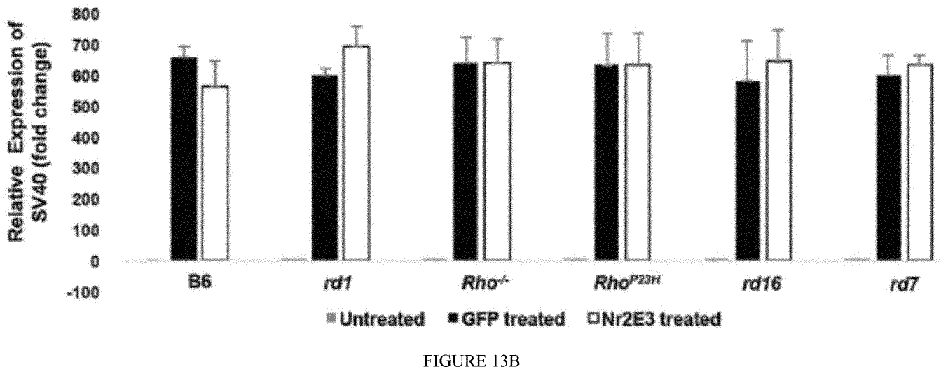

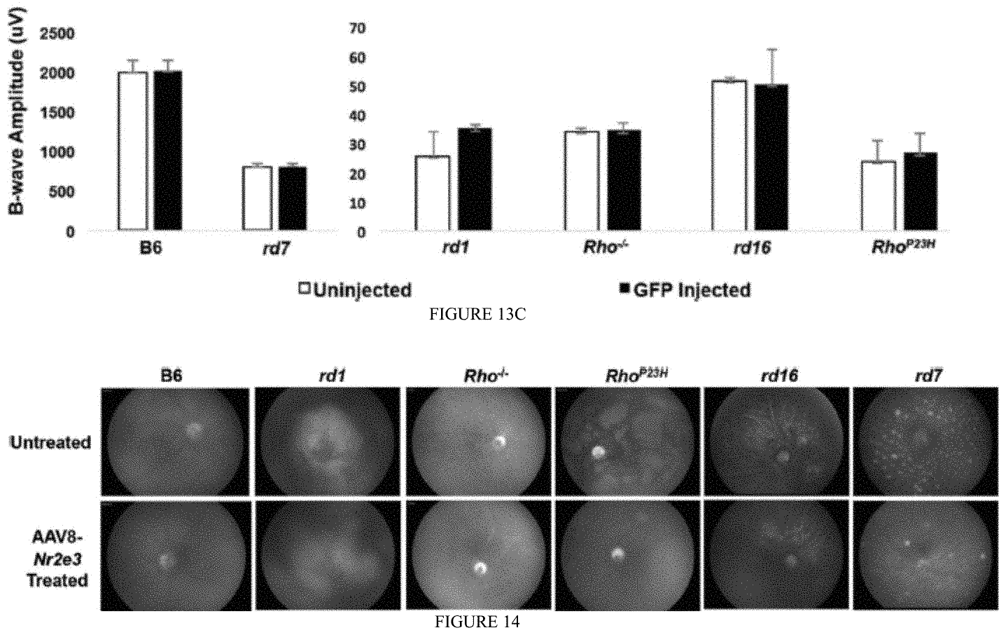

FIG. 13A shows Immunohistochemistry of AAV8-GFP (rd1, Rho−/−, RhoP23H, rd16, and rd7). All RP models except rd7 have only 0-1 cells in the ONL at P30 and GFP expression is more pronounced in other layers yet has no impact on disease.

FIG. 13B shows semiquantitative analysis of SV40 (part of AAV8) expression in untreated, AAV8-GFP, and AAV8-Nr2e3 retinas of B6 control and RP models relative to beta-actin.

FIG. 13C shows ERG B-wave amplitudes of uninfected and AAV8-GFP injected RP models and B6 control. Animals injected at P0, tissue collected at P30. Results are mean±SEM. N=7.

FIG. 14 shows AAV8-Nr2e3 rescues clinical phenotype in multiple mouse models of RP. Fundus of P0 injected AAV8-Nr2e3 treated and untreated animals evaluated at P30 (B6 and rd1) or P90-P120 (Rho−/−, RhoP23H, rd16, and rd7). N=7.

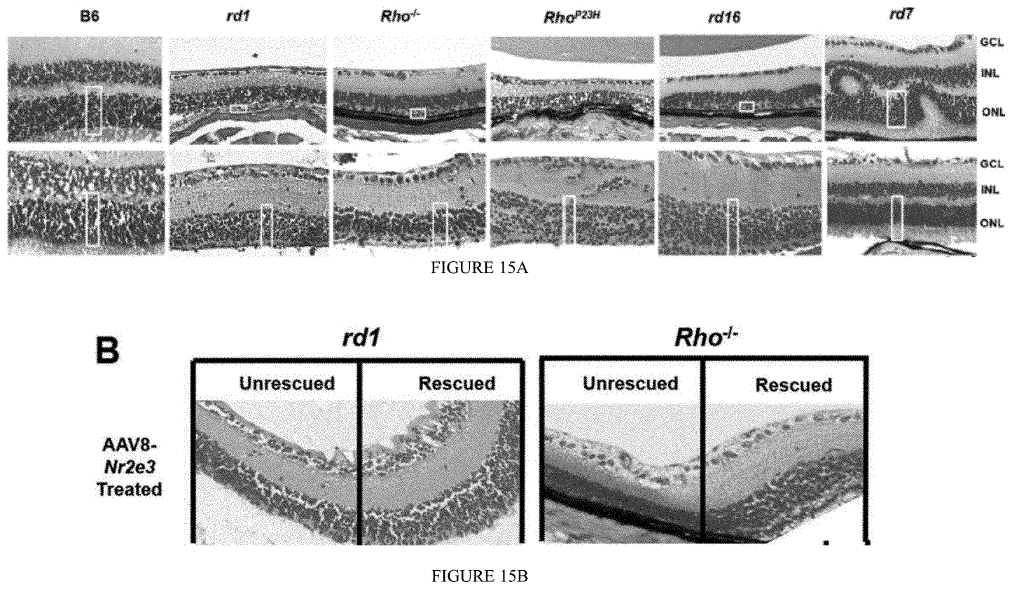

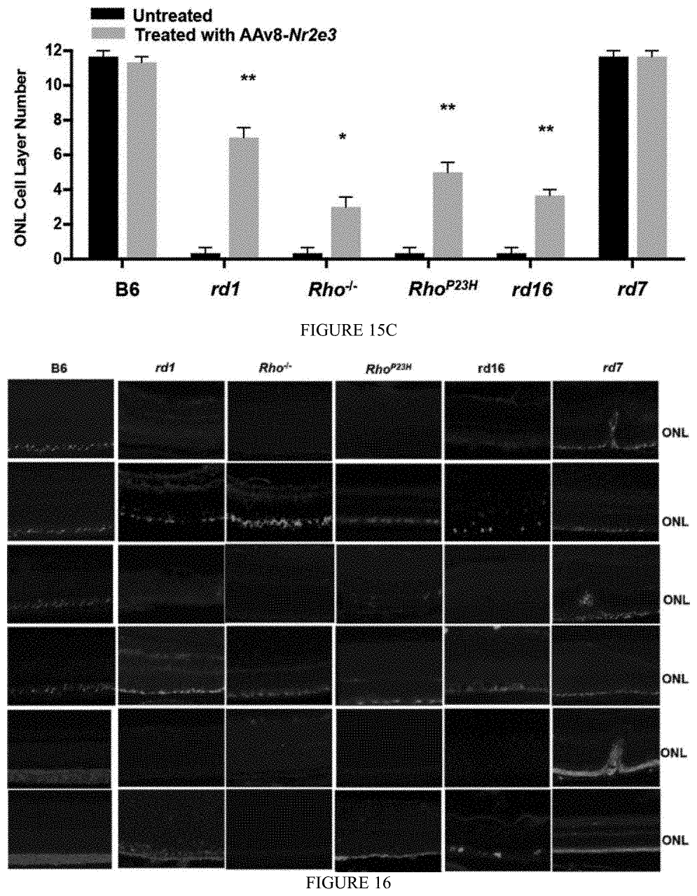

FIG. 15A is hematoxylin/eosin staining of AAV8-Nr2e3 treated (bottom row) and untreated (top row) retinas with white boxes indicating location of cell count.

FIG. 15B is hematoxylin/eosin staining showing rescued and un-rescued regions in retinas treated with AAV8-Nr2e3.

FIG. 15C is a bar graph showing cell layer numbers of ONL from AAV8-Nr2e3 treated and untreated animals in different RP models. Results are mean±SEM. N=7.

FIG. 16 shows immunohistochemistry of P0 injected AAV8-Nr2e3 treated and untreated retinas labeled with green opsin, blue opsin and rhodopsin evaluated at P30 (rd1) or P90-P120 (Rho−/−, RhoP23H, rd16, and rd7) and B6 control. N=7. From top to bottom row: untreated/green opsin; AAV8-Nr2e3 treated/green opsin; untreated/blue opsin; AAV8-Nr2e3 treated/blue opsin; untreated/rhodopsin; and AAV8-Nr2e3 treated/rhodopsin.

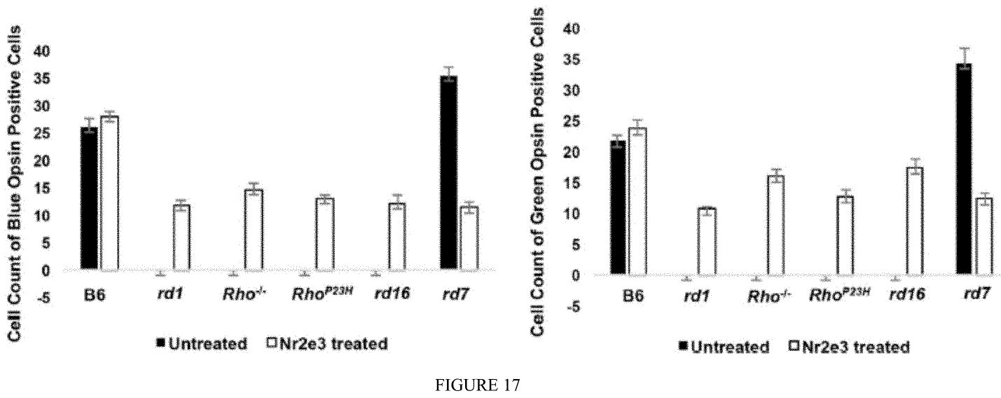

FIG. 17 is a bar graph showing semiquantitative analysis of cell counts of blue and green opsin-positive photoreceptor cells per 100 Results are mean±SEM. N=7.

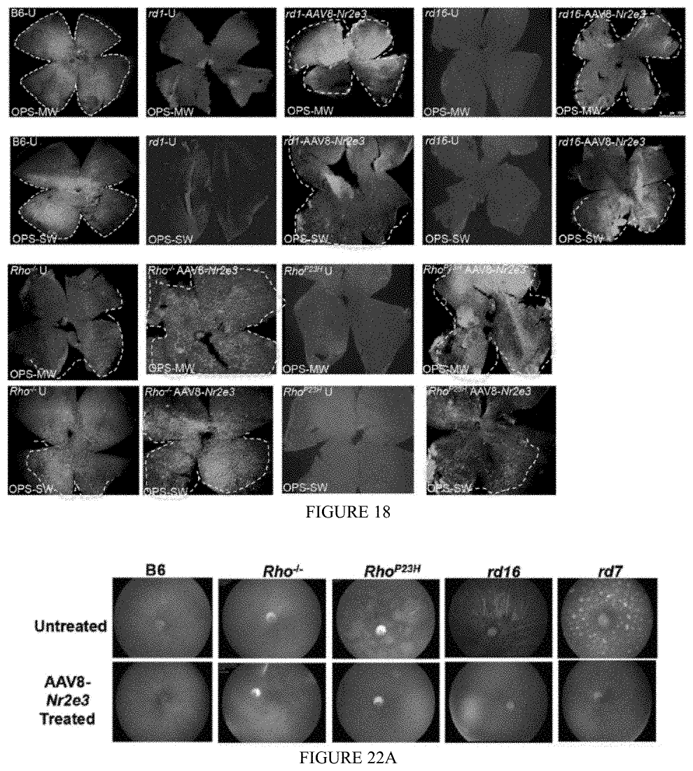

FIG. 18 shows evaluation of whole mounts of green opsin and blue opsin at 1-month old C57B16/J control, as well as 1-month rd1, Rho−/−, RhoP23H, and rd16 animals treated with AAV8-Nr2e3 at P0 and untreated animals. N=7.

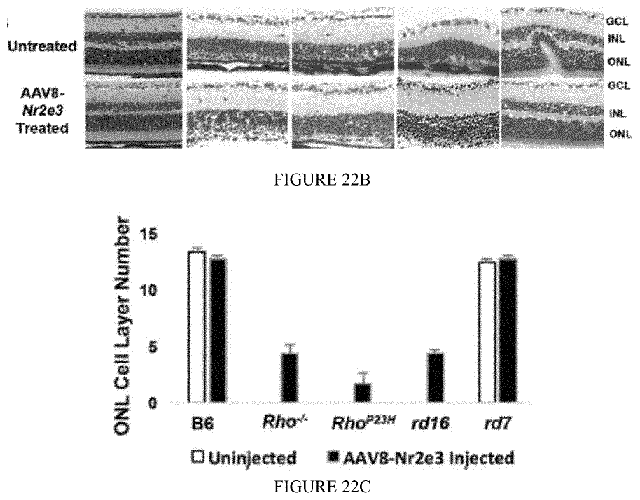

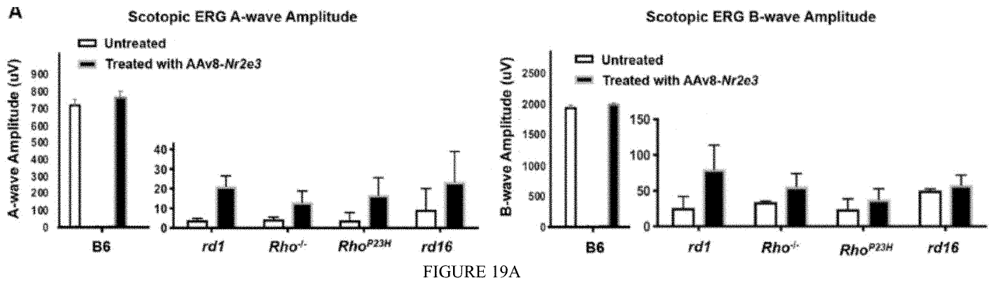

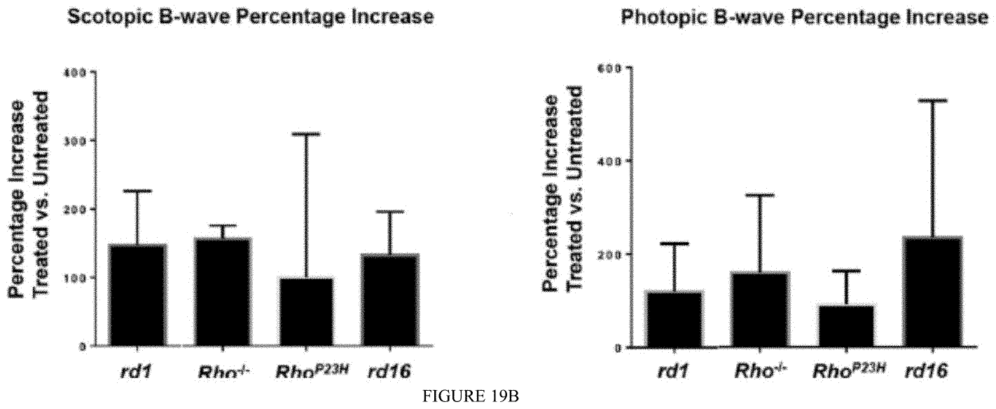

FIG. 19A shows scotopic and photopic ERG B-wave amplitudes evaluated at P30 (rd1) or P90-P120 (Rho−/−, RhoP23H, and rd16) for AAV8-Nr2e3 treated and untreated animals; B6 control ERGs shown.

FIG. 19B shows percent increase in ERG B-wave responses in the treated RP models. Results are mean±SEM. N=7.

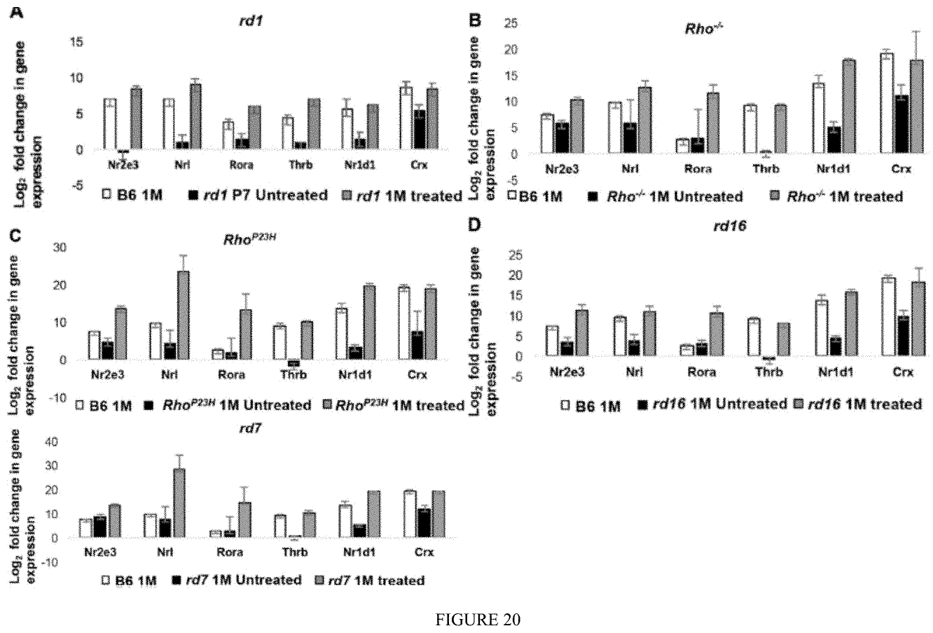

FIG. 20 shows relative expression levels of Nr2e3, Nrl, Rora, Thrb, Nr1d1, and Crx at P30 Nr2e3 treated mutant strains (rd7, Rho−/−, RhoP23H, and rd16) and rd1 at P7 compared with the corresponding untreated controls and normalized to beta-actin. Results are mean±SEM. N=7.

FIG. 21 is a table showing the rate of disease progression in RP models.

FIG. 22A shows fundus of animals (Rho−/−, RhoP23H, rd16, and rd7) injected with AAV8-Nr2e3 at P21 at 2-3 months post injection.

FIG. 22B shows hematoxylin/eosin staining of animals in FIG. 22A.

FIG. 22C shows a bar graph of cell layer numbers of outer nuclear layer between AAV8-Nr2e3 treated and untreated animals in the four RP models and B6 control. Results are mean±SEM. N=7.

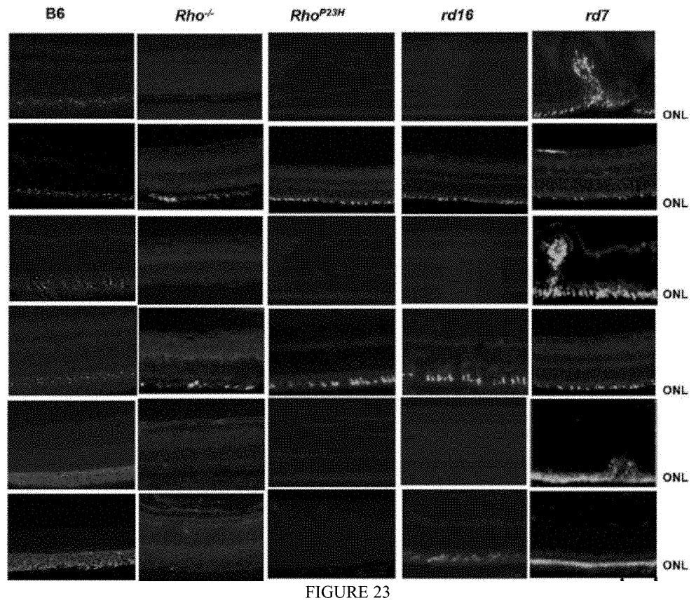

FIG. 23 is immunohistochemistry of green opsin, blue opsin and rhodopsin of treated and untreated animals (Rho−/−, RhoP23H, rd16, and rd7) evaluated at 2-3 months after injection of AAV8-Nr2e3. From top to bottom row: untreated/green opsin; AAV8-Nr2e3 treated/green opsin; untreated/blue opsin; AAV8-Nr2e3 treated/blue opsin; untreated/rhodopsin; and AAV8-Nr2e3 treated/rhodopsin.

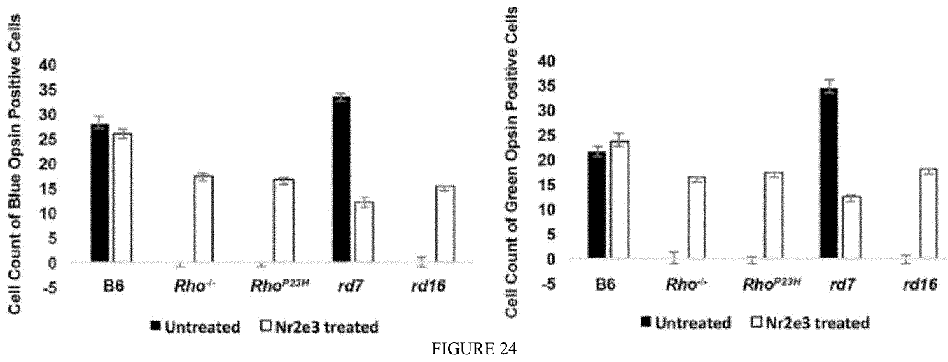

FIG. 24 is a bar graph showing semiquantitative analysis of cell counts of blue and green opsin-positive photoreceptor cells per 50 μm of the retina following AAV8-Nr2e3 treatment. Results are mean±SEM. N=7.

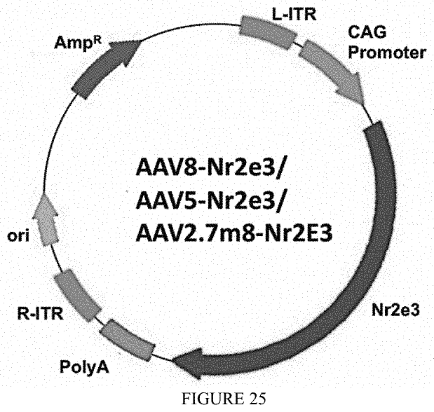

FIG. 25 is a schematic illustration of some of the plasmids of the present invention.

### DETAILED DESCRIPTION OF THE INVENTION

Genetic heterogeneity is observed for many Mendelian, single gene disorders including those resulting in ocular diseases or disorder. While environmental influences provide minor contributions, variations in phenotypic outcome are generally attributable to allelic heterogeneity or genetic modifier genes, allelic variants distinct from the mutant gene, which can affect disease onset, progression, and outcome by either increasing or reducing disease severity.

One aspect of the invention provides compositions and methods for a gene therapy that modify or restore the signaling pathways and/or function of photoreceptors for use in the treatment and prevention of ocular diseases or disorders. For example, one specific embodiment of the invention is directed to an Adeno-Associated Virus Serotype 5 capsid containing human Nuclear Hormone Receptor NR2E3 (AAV5-hNR2E3) gene therapy for the treatment of NR2E3 mutation associated recessive retinal degenerative diseases.

Mutations in over hundreds of genes are associated with inherited retinal degenerations (see, for example, sph.uth.edu/retnet/). Mutations in the nuclear receptor gene, NR2E3, have been identified in patients with autosomal recessive retinal degeneration with various phenotypic features. Clinical diagnostics prior to molecular clarity led to a multitude of names given to this genetic disease. Based mainly on ophthalmoscopic examinations, patients were categorized as having retinitis pigmentosa, clumped pigmentary retinopathy, or vitreo-retinal degeneration with cystoid maculopathy and retinoschisis, among other descriptions. A prevalence rate for NR2E3 mutation associated recessive retinal degenerations may be estimated from reports of causal genotypes of non-syndromic retinitis pigmentosa (RP). It can vary from 0.25% of recessive RP to nearly 3% from an Asian population. It is thus regionally dependent. One must also be cautious that any estimate does not include autosomal dominant NR2E3 patients who have a different set of disease manifestations and presently would not be targets of the gene augmentation/replacement strategy proposed herein.

The unifying phenotypic feature of autosomal recessive mutations in human NR2E3 is a common mechanism including excess and hypersensitivity of short-wavelength cones (S-cones) with reduced long and middle-wavelength (L/M) cone retinal function and little or no rod photoreceptor function. The disease mechanism was identified by clinical testing in 1990 and confirmed in many subsequent reports. The NR2E3 gene and causative mutations were discovered in 2000. Without being bound by any theory, it is believed that the unique physiological features result from a developmental aberration in cone proliferation, but the disease is not stationary and is accompanied by a progressive retinal degeneration leading to severe visual disability.

Nuclear hormone receptors (NHRs) play a critical role in modulating cellular homeostasis by regulating basic biological processes including development, metabolism, circadian cycle, and energy homeostasis. It has been shown that NHRs such as Nr2e3, Nr1d1, Rora, and Nr2c1 are important modulators of retinal disease. Nuclear Receptor Subfamily 2, Group E, Member 3 (NR2E3), first reported as photoreceptor-specific nuclear receptor (PNR), function either through ligand or co-factor activation to modulate gene expression of various rod and cone specific genes. NR2E3 has been implicated in regulating several key biological gene networks including development, metabolism, cell survival, apoptosis and energy homeostasis to regulate proper development of and maintenance of photoreceptors.

The retinal degeneration 7 (C57BL6/J_{rd7/rd7}, rd7) mouse lacks a functional Nr2e3 and serves as a model to study and evaluate therapies for NR2E3 associated retinal diseases 9, rd7 mice exhibit a significant increase of S-cones and progressive degeneration of rod and cone photoreceptor cells. Studies have demonstrated the efficacy of Nr2e3 gene augmentation as a therapy to ameliorate retinal disease. rd7 mice dosed with AAV5-mNr2e3 through intravitreal (IVT) and sub-retinal routes show clinical, histological, and molecular rescue of retinal degeneration. Furthermore, the NR2E3 mechanism of action is to function as a key regulator of several developmental, cellular and metabolic gene-networks.

One particular composition of the invention is an adeno-associated virus serotype 5-based vector comprising the human NR2E3 gene expression cassette containing: a) AAV2 ITR; b) the cytomegalovirus (CMV) enhancer; c) the chicken beta actin (CBA) promoter; d) chimeric intron; e) the cloned cDNA coding for human NR2E3 protein (44692 dalton); and f) the SV40 polyadenylation (PolyA) region. See, FIG. 1. SEQ ID NO:1 shows the sequence of the DNA corresponding to known normal human NR2E3 sequence (NCBI: NM_014249.3). The coding region for the hNR2E3 protein is from 197-1429. The protein sequence of NR2E3 is shown in SEQ ID NO:2.

One particular composition of the invention (see Examples section) is a product developed for a gene therapy utilizing NHRs, which have long been known to play a critical role in modulating cellular homeostasis by regulating basic biological processes including development, metabolism, circadian cycle, and energy homeostasis. One particular embodiment of the present invention is directed to a composition comprising an NHR gene (such as Nr2e3, Nr1d1, and Rora) to treat ocular or retinal diseases or disorders including RP as well as other degenerative diseases such as AMD. In one particular embodiment, the NHR gene is comprised of Nr2e3, a NHR gene expressed in adeno-associated viral vector that can be used as a gene therapeutic for the treatment of retinal degenerative diseases including subsets of RP. It should be appreciated, however, the scope of the invention includes recombinant DNAs, oligonucleotides, and recombinant adeno-associated virus (rAAV) vectors that include other genes disclosed herein such as those listed in the odd numbered sequence in SEQ ID NOs:1-69.

Compositions of the invention can also be used in other therapeutic indications related to ocular diseases or disorders. It has been shown that Nr2e3 is a dual activator/repressor and member of the NHR family and that, with other transcription factors, modulates cell fate and differentiation of rod and cone photoreceptor cells. In particular, Nr2e3 regulates cone cell proliferation in retinal progenitors and promotes rod differentiation in post-mitotic differentiating rod photoreceptors (FIG. 2) by suppressing cone genes while activating rod-specific genes. Nr2e3 has been shown to be one of the key factors in regulating retinal progenitor cells to produce the appropriate number of blue cones and also in directing proper rod cell differentiation. Delivery of Nr2e3 efficiently ameliorated clinical, morphological, and functional defects associated with retinal degeneration in a mouse model lacking functional Nr2e3. It has also been demonstrated that the mechanism of rescue at the molecular and functional level is at least in part through the re-regulation of key genes within the Nr2e3-directed transcriptional network. Without being bound by any theory, it is believed that these studies suggest that Nr2e3 can at least partially or fully rescue receptor disease of Infantile Refsum disease (IRD). IRD, also called infantile phytanic acid storage disease, is a rare autosomal recessive congenital peroxisomal biogenesis disorder within the Zellweger spectrum. This peroxisomal disorder typically presents in the first year of life with both systemic and ocular features. Night blindness is the major ocular feature and at least some have optic atrophy similar to the adult form.

Compositions and methods for gene therapies disclosed herein, in particular NHR gene therapy, provide a treatment that can restore retinal integrity and function across a range of genetically diverse IRDs and other degenerative retinal diseases. NHR gene therapy encompasses the targeted delivery and expression of certain NHRs that are expressed naturally in retinal tissue. It has been shown to rescue many genetic defects and can lead to vision-sparing therapies for rare IRDs such as enhanced S-cone syndrome, Goldman-Favre syndrome and RP, as well as other forms of retinal and macular degeneration.

Gene therapy using compositions and methods of the invention are capable of modifying disease states in the retina. Accordingly, compositions and methods of the invention provide therapeutic options with broad applicability. In one particular embodiment, therapeutic NHRs have been identified for their ability to modify disease progression through the regulation of key gene networks that can restore retinal homeostasis and rescue the defects caused by inherited gene mutations. The use of genetic modifiers represents a powerful and remarkably broadened means of treating a variety of retinal degenerative diseases, as compared to single-gene replacement therapy. While single-gene replacement therapies have shown tremendous promise in rare retinal diseases, they are highly specific and cannot ameliorate a multitude of disease-causing genetic defects. On the other hand, NHRs play a vital role in regulating retinal cell development, maturation, metabolism, visual cycle function and survival. See, for example, Olivares et al. in Scientific Reports, 2017, 690 (Scientific Reports|7: 690|DOI: 10.1038/s41598-017-00788-3)

Disease outcome is a result of a primary mutation as well as modifier alleles.

Nr2e3 is believed to be a master regulator of several key pathways in retinal development and function. Nr2e3 potentially prevents and attenuates disease by resetting the homeostatic state of key gene networks in the presence of a primary mutation (FIG. 3).

Nr2e3 regulates multiple transcriptional networks, such as cell survival, metabolism, inflammation and phototransduction, that impact RP. Nr2e3 and Nr1d1 are cofactors that modulate many of the same gene networks. It has been demonstrated preclinically that Rora offers a protective allele in AMD where loss of photoreceptor cells leads to blindness. Nr2e3 regulates the expression of both Nr1d1 and Rora. Thus, the nuclear receptors work in overlapping networks to modulate normal retinal development and function. These receptors impact gene expression of hundreds of genes and numerous networks and, as such, may be potent modifiers of retinal disease and degeneration.

While there are some gene replacement clinical trials in progress, these treatments only address a few known RP genes and rely on identifying the primary mutation, which is not possible for approximately 40% of all RP patients. Additionally, the severity and progression of RP disease is greatly impacted by the genetic background in which the mutation is present. In contrast, compositions and methods of the present invention are applicable in treating substantially all RP patients as an entire gene sequence of Nr2e3 can be used.

Nr1d1, an important NHR gene, regulates many processes, such as differentiation, metabolism and the circadian rhythms. Recently, various preclinical studies demonstrated a role for Nr1d1 in the retina. Nr1d1 forms a complex with Nr2e3, CRX and NRL, key transcriptional regulators of retinal development and function. Importantly, Nr1d1 binds the Nr2r3 protein directly and acts synergistically to regulate transcription of photoreceptor-specific genes. Thus, by using Nr1d1 gene, compositions and methods of the present invention can be used to modify the effects of Nr2e3-associated retinal degeneration (FIGS. 4 and 5).

IRDs are caused by genetic mutations that are passed down within families and lead to progressive disease, severe visual impairment and blindness. Treating these conditions has been a significant challenge due to the sheer volume of potential therapeutic gene targets. Gene replacement therapy is a promising approach to provide a sustained restoration effect of normal retinal function for a mutated gene, but such therapies can only address one gene at a time, limiting their effectiveness. Developing a custom gene therapy for genetic defects in each of the more than 200 known genes linked to RP would not only be expensive but also may not be possible due to size, class, or localization that will impact delivery of the gene. Not all genes and disease expressions are amenable to gene therapy, and for the approximately 40% of patients whose genetic mutations remain unknown, there are few or no therapeutic options.

In contrast, compositions and methods of the present invention can ameliorate multiple forms of RP without requiring knowledge of the mutated gene, and provides feasibility of treatment for substantially all RP patients.

RP is a group of heterogeneous, pleiotropic IRDs that affect approximately one in every 4,000 individuals. Currently, there is no cure for RP and over 40% of RP cannot be genetically diagnosed. RP is heterogeneous and varies greatly in age of onset, rate of progression, and even genetic etiology, yet a common pathology of photoreceptor (PR) cell degeneration develops. In addition to RP, no effective treatments are available for a large number of other retinal degenerative diseases including treatments specifically for dry AMD.

Another embodiment of the invention includes using a transferrin gene for treating ocular diseases or disorders, such as choroidal neovascularization (CNV). CNV refers to the uncontrolled growth of choroidal vasculature which can lead to severe vision loss in diseases such as pseudoxanthoma elasticum, angioid streaks, histoplasmosis, punctuate inner choroidopathy and wet age-related macular degeneration (AMD). Wet AMD occurs when the deposition of drusen (complement components, lipids, and apolipoproteins) causes confined ischemic regions resulting in hypoxia. It is believed that hypoxia leads to an increase in the secretion of vascular endothelial growth factor (VEGF), which activates choroidal endothelial cells to secrete matrix metalloproteinases (MMP). Metalloproteinases degrade the extracellular matrix, thereby allowing for the proliferation of endothelial cells and their migration towards the retina. The effect of MMP eventually results in the development of new blood vessels, or CNV, which can cause retinal detachment and hemorrhage and the formation of sub retinal lesions due to blood and lipid leakage. Once manifested, CNV is a major cause of vision loss in the elderly population of industrialized nations.

Treatment of CNV is currently limited to a fraction of the patient population and focuses on restraining the detrimental role of VEGF in vascular hyperpermeability and new blood vessel formation. However, VEGF also plays a constructive key role in physiological activities such as wound healing, photoreceptor survival, and maintaining the choroid capillary bed. Currently, Ranibizumab (Lucentis™), Aflibercept (Eylea™) and pegaptanib (Macugen™) are the only two therapeutic agents that have been approved to date to treat CNV. These agents inhibit VEGF. It has been shown that ranibizumab is generally more effective than pegaptanib in treating CNV. Ranibizumab binds to all isoforms of VEGF-A and inhibits VEGF activity including vascular permeability and growth. Other than the two mentioned therapeutic agent, bevacizumab (Avastin™), the parent full length antibody of ranibizumab, is also being explored as an off label treatment for CNV.

Despite the success of these therapies in treating CNV there are inherent drawbacks in these therapies, including lack of apoptosis in activated endothelial cells, and potential impairment of VEGF related physiological activities such as wound healing. In addition, use of ranibizumab leads to systemic risks including increased rate of thromboembolic events after intravitreal administration in humans. Intravitreal bevacizumab has also been associated with ischemic attack, blood pressure elevation, cerebrovascular accidents, and death. Further, in a clinical trial with patients suffering from CNV, the response rate to ranibizumab was only ˜40% in patients with CNV and the gain in number of letters was only 7.2.

In some embodiments, the recombinant DNA and/or the recombinant adeno-associated virus (rAAV) vector of the invention includes a fusion gene encoding transferrin-tumstatin protein. Such compositions can be used in a gene therapy to treat, for example, CNV and other ocular diseases or disorders. It should be appreciated that rAAV vector of the invention can also include a fusion gene encoding various functional counterpart of a defective gene associated with manifestation an ocular condition or disease in combination with one or more of the various therapeutic gene(s) disclosed herein. Some of the functional counterpart of a defective genes are disclosed in the odd numbered sequences in SEQ ID NOs:1-69.

In some embodiments, the gene therapy of the invention is used to increase the level of particular protein (e.g., even numbered sequences in SEQ ID NOs:2-70) to ameliorate, prevent, or treat an ocular condition or disease. As used herein, an “increase” in a level or activity of a protein (e.g., a nuclear hormone receptor), a downstream signaling component (e.g., phototransducin), or a photoreceptor can be measured by methods known in the art, such as RT-PCR, Western blot, transactivation assays, or electroretinography. An increase in expression level or activity can be 1%, 2%, 5%, 10%, 25%, 50%, 75%, 1-fold, 2-fold, 5-fold, or 10-fold increase when compared to expression level or activity before treatment, or to expression level or activity in subjects that are suffering from the ocular disease or disorder that have not received treatment. Similarly, and as described herein, a “decrease” in a level or activity of a protein (e.g., nuclear hormone receptor), a downstream signaling component (i.e., phototransducin), or a photoreceptor can be measured by methods known in the art, such as RT-PCR, Western blot, transactivation assays, or electroretinography, can be measured by methods known in the art, such as RT-PCR or transactivation assays. A reduction in expression level or activity can be 1%, 2%, 5%, 10%, 25%, 50%, 75%, 1-fold, 2-fold, 5-fold, or 10-fold reduced when compared to expression level or activity before treatment, or to expression level or activity in subjects that are suffering from the ocular disease or disorder that have not received treatment.

The subject can be any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In some embodiments, composition of the invention reduces the expression or activity of a cone photoreceptor specific transducin, wherein the cone photoreceptor specific transducin comprises Gnat2. Alternatively or in addition, the composition of the invention reduces the expression or activity of an S-cone-specific opsin, wherein the S-cone specific opsin comprises Opn1sw.

It should be appreciated that whether the treatment increases or decrease the level of a particular protein depends on whether the ocular clinical condition or disease is due to decrease or increase in the level of that protein, respectively. In general, the ocular condition or disease is due to decrease in the level of “normal” or non-mutant protein that is expressed by the gene of interest.

A suitable nucleic acid sequence of human Nr1d1 is set forth in SEQ ID NO: 5 or a fragment thereof. A suitable nucleic acid sequence of human Nr2e3 is set forth in SEQ ID NO: 1 or a fragment thereof. A suitable nucleic acid sequence of human Rora is set forth in SEQ ID NO: 7 or a fragment thereof. A suitable nucleic acid sequence of human Nupr1 is set forth in SEQ ID NO: 9 or a fragment thereof. A suitable nucleic acid sequence of human Nr2c1 is set forth in SEQ ID NO: 11 or a fragment thereof. Other nucleic acid sequence for various genes are set forth in odd numbered sequences in SEQ ID NOs: 1-69 along with the corresponding proteins in even numbered sequences in SEQ ID NOs: 2-70, respectively.

The composition comprising of the invention can be administered via adeno-associated virus-based gene delivery. However, genes or the oligonucleotide can also be administered via electroporation, via biodegradable Nile red poly(lactide-co-glycolide) (PLGA) nanoparticle-based gene delivery, small molecule-based gene delivery, naked DNA delivery, or genome editing systems, e.g., CRISPR.

Typically, the polynucleotides (e.g., recombinant DNA or rAAV vector) are purified and/or isolated prior to administration. Specifically, as used herein, an “isolated” or “purified” nucleic acid molecule is substantially free of other chemical precursors or other chemicals when chemically synthesized. Purified compounds (e.g., recombinant DNAs or rAAV vectors) are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, high-performance liquid chromatography (HPLC), or mass spectroscopy analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide that has been separated from the components that naturally accompany it. Typically, the nucleotides are substantially pure when they are at least 60%, 70%, 80%, 90%, 95%, or even 99%, by weight, free from the proteins and naturally-occurring organic molecules with they are naturally associated.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company, incorporated herein by reference.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide.

Although the phrase “nucleic acid molecule” primarily refers to the physical nucleic acid and the phrase “nucleic acid sequence” refers to the linear list of nucleotides of the nucleic acid molecule, the two phrases can be used interchangeably.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent ocular disease in a subject. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, e.g., ocular disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

“Recombinant vector” refers to a vector that includes a heterologous nucleic acid sequence which is capable of expression in vivo.

The term “transgene” refers to a polynucleotide that is introduced into a cell and is capable of being transcribed, translated, and/or expressed under appropriate conditions leading to a desired therapeutic outcome.

“Genome particles (gp),” or “genome equivalents,” as used in reference to a viral titer, refer to the number of virions containing the recombinant AAV DNA genome, regardless of infectivity or functionality. The number of genome particles in a particular vector preparation can be measured by procedures such as described in Clark et al., Hum. Gene Ther., 1999, 10, pp. 1031-1039; and Veldwijk et al., Mol. Ther., 2002, 6, pp. 272-278, all of which are incorporated herein by reference in their entirety.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. 1973, Virology, 52:456, Sambrook et al. 1989, Molecular Cloning, a laboratory manual, Cold Spring Harbor Laboratories, New York, Davis et al., 1986, Basic Methods in Molecular Biology, Elsevier, and Chu et al., 1981, Gene 13:197, all of which are incorporated herein by reference in their entirety. Such techniques can be used to introduce one or more exogenous DNA moieties into suitable host cells.

The term “heterologous” means sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term DNA “control sequences” refers those sequences that are needed for replication, transcription, and/or translation. Thus, the term refers collectively to promoter sequences, polyadenylation signals, transcription termination sequences, upstream regulatory domains, origins of replication, internal ribosome entry sites (“IRES”), enhancers, and the like. However, it should be noted that not all of these control sequences need always be present so long as the selected coding sequence is capable of being replicated, transcribed and translated in an appropriate host cell.

The term “promoter” refers to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

The term “operably linked” refers to an arrangement of elements wherein the components are configured to perform their usual function. Thus, control sequences operably linked to a coding sequence are capable of effecting the expression of the coding sequence. It should be appreciated that the control sequences need not be contiguous with the coding sequence, so long as they function to direct the expression thereof. For example, intervening untranslated yet transcribed sequences can be present between a promoter sequence and the coding sequence and the promoter sequence can still be considered “operably linked” to the coding sequence.

The term “modulate” means to vary the amount or intensity of an effect or outcome, e.g., to enhance, augment, prevent, diminish, reduce or eliminate.

The terms “ameliorate” and “alleviate” are used interchangeably herein and mean to reduce or lighten. For example, one may ameliorate the symptoms of a disease or disorder by making the disease or symptoms of the disease less severe.

The terms “therapeutic,” “effective amount” and “therapeutically effective amount” are used interchangeably herein and refer to a sufficient amount of the composition or agent to provide the desired response, such as the prevention, delay of onset or amelioration of symptoms in a subject or an attainment of a desired biological outcome.

“Treatment” or “treating” a particular ocular condition or disease includes: (1) preventing the ocular condition or disease, i.e. preventing the development of the ocular condition or disease or causing the ocular condition or disease to occur with less intensity in a subject that may be exposed to or predisposed to the disease but does not yet experience or display symptoms of the ocular condition or disease, (2) inhibiting the ocular condition or disease, i.e., arresting the development or reversing the ocular condition or disease state, or (3) relieving symptoms of the ocular condition or disease, i.e., decreasing the number of symptoms experienced by the subject, as well as changing the cellular pathology associated with the ocular condition or disease.

It should be appreciated that the present invention is not limited to particular formulations or process parameters disclosed herein. Although a number of methods and materials similar or equivalent to those described herein can be used in the practice of the present invention, for the sake of brevity and clarity only a few representative materials, methods, and protocols are described herein.

The present invention utilizes a rAAV containing (i) a therapeutic gene for treating an ocular disease or condition, (ii) a normal gene of a defective gene that causes the ocular disease or condition, iii) or both. These genes are disclosed herein and includes those shown in the odd numbered sequences in SEQ ID NOs:1-69.

The constructs described herein, are delivered to the subject in need of a treatment for an ocular condition or disease using any of several rAAV gene delivery techniques that are known to one skilled in the art. For example, genes can be delivered either directly to the subject or, alternatively, delivered ex vivo, to appropriate cells, such as cells derived from the subject, and the cells reimplanted in the subject.

Various AAV vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med (1994) 179:1867-1875.

Some embodiments of the invention are directed to nucleic acids that encode a biologically active fragment or a variant of Nr1d1, Nr2e3, Rora, Nupr1, or Nr2c1. A biologically active fragment or variant is a “functional equivalent”, a term that is well understood in the art.

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Example 1: AAV2.8-mNr2e3 gene augmentation therapy prevents retinal degeneration in rd7 mice: AAV2.8-mNr2e3 gene delivery in neonatal rd7 mice prevented retinal degeneration. rd7 retinas injected at P0 and evaluated at 3 months of age demonstrated sustained rescue as measured by changes in the fundus, morphology, and expression of opsin genes (FIG. 6). Comparative analyses of fundus images of untreated and AAV2.8-Nr2e3 (1×10⁹ genome copy (gc)/0.5 μl/eye) treated eyes demonstrated the reduction in disease phenotypes in treated animals, such as decrease in numbers of pan-retinal spotting, rosettes and whorls. Histology and immunochemistry of retinal sections indicated complete reversal of retina dysplasia and restoration of normal morphological features of retinal layers in treated eyes in contrast to untreated ones. Immunochemistry also indicated the uniform, homogenous expression of blue opsin, green opsin, and rhodopsin proteins in photoreceptor layers. A reset of over 40 genes was observed in seven Nr2e3-regulated gene networks indicating the mechanism of rescue is through re-establishing a homeostatic state of the retina (FIG. 7).

Example 2: AAV2.8-Nr2e3 rescues retinal degeneration in early-intermediate stage of rd7 disease: AAV2.8-Nr2e3 gene delivery in early-intermediate stage of rd7 retinal degeneration reversed retinal disease. rd7 retinas injected at P21 and evaluated at 3 months of age showed reversal of retina spots and retinal dysplasia (FIG. 8). Fundus and histology showed loss of retinal spotting and whorls, complete reversal of retina dysplasia in treated eyes compared to untreated retinas. Immunochemistry confirmed the uniform, homogenous expression of blue opsin, green opsin, and rhodopsin proteins and restoration of normal photoreceptor layers morphology.

Example 3: AAV5-mNr2e3 reverses retinal degeneration in rd7 mice following subretinal injection and intravitreal (IVT) injections at early-intermediate stage of disease: An AAV5-mNr2e3 construct was generated using standard triple plasmids (murine Nr2e3, helper, Rep2/Cap5) transfection of HEK-293 cells and purification using density gradient ultracentrifugation process. See Example 4 below. AAV5.mNr2e3 (110⁹ gc/0.5 μl/eye) was administered in 3-month-old rd7 mice, when retinal dysplasia and disease manifestation at an intermediate stage. Dosing at 3-months of age was to more closely mimic disease stage when patients might receive treatment. In this study, effect of dosing routes (IVT vs subretinal) was also assessed on delivery and efficacy. Preliminary data showed that AAV5-mNr2e3 therapy both by IVT and subretinal route effectively reversed clinical (loss of retinal spots) and histological (loss of whorls and rosettes) manifestations and confirmed opsin expression in rd7 mice was restored to a normal level (FIGS. 9, 10, and 11). Green fluorescent protein (GFP) expression confirmed gene delivery to the retina. Based on analyses of data, the efficacy of AAV5-mNr2e3 between IVT and subretinal route of administration appeared to be similar, although it is not quantitative. These data demonstrate that Nr2e3 can be a potent gene therapeutic to treat Nr2e3 associated recessive retinal degenerative diseases in human.

Example 4. An AAV5-mNr2e3 construct was generated using standard triple plasmids (murine Nr2e3, helper, Rep2/Cap5) transfection of HEK-293 cells and purification using density gradient ultracentrifugation process. Briefly, hNR2E3 transgene cassette plasmid was designed, synthesized and produced in bacterial cells. Plasmid was characterized for identity, and integrity of various regulatory (AAV2-ITR, CMV enhancer, CBA promoter and chimeric intron from rabbit globulin gene, and SV40 poly A) elements. Rep sequence was chosen from wild type AAV2 serotype while cap gene sequence was chosen from AAV5 wildtype serotype. Helper plasmid was used to provide various factors for AAV5 production in adherent HEK-293 cells. Briefly, HEK-293 cells were expanded on Cellstack™ culture dishes and transfected with helper, rep/cap and transgene plasmids. Following production, cells were lysed and the product was purified using density gradient ultracentrifugation process. Transgene copy number (vg/mL) in the purified product was determined using qPCR methods. Purified product was stored at −70° C. and used for pre-clinical in vitro and in vivo POC studies.

Example 5. AAV8 Nr2e3 cloning and preparation: AAV8-Nr2e3 vector was generated at the Gene Transfer Vector Core, Grousbeck Gene Therapy Center, Mass Eye and Ear (vector.meei.harvard.edu). Briefly, HEK293 cells were transfected with the AAV8 rep-cap packaging, Ad-helper, and AAV2 ITR-flanked transgene constructs. After 3 days, cells and media were harvested in high salt conditions, treated with Benzonase, and cellular debris was precipitated. The supernatant was subjected to tangential flow filtration and retentate was subsequently run over an Iodixanol ultracentrifugation density gradient. AAV fractions were collected and buffer exchange was performed for final formulation in phosphate buffered solution (PBS)+5% glycerol. The ubiquitous CAG promoter was used in the vector. CAG is a strong synthetic hybrid promoter consisting of the cytomegalovirus enhancer fused to the chicken beta-actin promoter. Mouse Nr2e3 cDNA to be packaged into AAV8 was generated by RT-PCR from mRNA of a B6 mouse retina using the following primers: forward: GCTGTACAAGGGCGGA TGAGCTCTACAGTGGCT (SEQ ID NO:75); reverse: ATACCGGTTGG CACTCCCAACTAGTT (SEQ ID NO:76). These primers were introduced at the restriction sites BsrGI at the 5′ end and Agel at the 3′ end of Nr2e3 cDNA, and were used for cloning into the pZac2.1-CASI-eGFP-RGB plasmid (also known as pAAV). Final products were verified by restriction enzyme digestions and sequencing.

Example 6. AAV5-Nr2e3-GFP and AAV2.7m8-Nr2e3 cloning and preparation: The AAV2.7m8 plasmid was obtained from Addgene (Addgene plasmid #64839: n2t.net/addgene:64839; RRID: Addgene_64839) (see, for example, FIG. 25). AAV5-Nr2e3-GFP and AAV2.7m8-Nr2e3 were cloned and constructed by VectorBioLabs (Malvern, Pa., USA), similar to the method described above for AAV8-Nr2e3. Mouse Nr2e3 was introduced into restriction sites NheI and KpnI of AAV5 and restriction sites EcoRI and Xhol of AAV2.7m8 using the following primers: Forward: CCTAAGCTTATGAGCTCTACAGTGGCT GCCTCC (SEQ ID NO:77) Reverse: ATCGAATTCGGATCCGGTACCCTAGTTT TTGAACATGTCACACAG (SEQ ID NO:78). The final product was verified by restriction enzyme digest and sequencing.

Example 7. Subretinal injection: All AAV-Nr2e3 constructs were delivered by subretinal injection. Control injections included no injection in the contralateral eye, untreated animals, and GFP only injections. Approximately 600 experimental animals were used in this study. No gender bias was observed and both males (48.94%) and females (51.06%) were used equally in the study. P0 pups were anesthetized on ice, and the eyelids were carefully opened along the eyelid fissure using a 30 gauge (G) needle. The 30 G needle was then used to create a hole in the sclera adjacent to the limbus, and a blunt 33 G cannula attached to a Hamilton syringe was advanced into the eye. A slight resistance to the needle indicated Bruch's membrane was reached. A total of 1×10⁹ viral genomes (vg) in a total volume of 0.5 μL was manually injected slowly and gently into the subretinal space of the adult or P0 mice. Subretinal injection was performed in adults as described above after anesthetizing animals by intraperitoneal (IP) injection with a mixture of ketamine (1 mg/mL) and xylazine (0.4 mg/mL).

Example 8. Clinical examination: Fundus examination and optical coherence tomography, (OCT) were performed on adult injected and uninjected animals. Animals were anesthetized with a mixture of ketamine (1 mg/mL) and xylazine (0.4 mg/mL) and pupils were dilated with 1% tropicamide. Fundus images were taken using the Micron III Retinal Imaging Camera and Stream Pix software (Phoenix Research Laboratories, Pleasanton, Calif., USA). Following fundus imaging, OCT was performed using the Bioptigen OCT scanner and software. Mice were restrained in a mounting tube and the fundus camera in the optical head of the apparatus and alignment was guided by monitoring and optimizing the real time OCT image of the retina. Four rotational cross section scans (dorsal-ventral and nasal-caudal) with 100 series/scan were taken for each retina. Data were analyzed using Bioptigen OCT software (N=10/strain/experimental group).

Example 9. Electroretinography: Electroretinography (ERG) analysis was performed on Nr2e3 treated and untreated animals. Briefly, mice were anesthetized with an IP injection of 1 mg/mL ketamine and 0.4 mg/mL xylazine in a saline carrier (10 mg/g of body weight), and mouse eyes were dilated with 1% tropicamide and 2.5% phenylephrine hydrochloride applied topically. Dark- and light-adapted ERGs were performed using the Espion Visual Electrophysiology System (Diagnosys, Littleton, Mass.) with gold loop electrodes (Diagnosys LLC) placed on the apex of the cornea. A reference needle electrode was inserted subcutaneously in the forehead and a ground electrode was placed subcutaneously at the base of the tail. For scotopic recordings, mice were dark adapted for at least 6 h and then anesthetized before recording. Dark-adapted responses were recorded to short wavelength (λmax=470 nm; Wratten 47A filter; Kodak, Rochester, N.Y.) flashes of light over a 4.0-log unit range of intensities (0.3-log unit steps). Light-adapted responses were obtained with white flashes (0.3 log unit steps) on a rod-saturating background after 10 min of exposure to the background light to allow complete light adaptation. Signal processing was performed using EM for Windows v7.1.2. Signals were sampled every 0.8 ms over a response window of 200 ms (LKC Technologies, Inc., Gaithersburg, Md.). Responses were averaged for each stimulus condition with up to 50 records for the weakest signals. Dark-adapted responses and light-adapted responses illustrated in this study were obtained using stimulator intensities of 24.1 cd s/m² for scotopic responses and 25.6 cd s/m2 for photopic responses (N=7/strain/experimental group).

Example 10. Histology: Following euthanasia, eyes were cauterized to mark dorsal orientation, and enucleated. Tissue samples were collected and immediately immersed in freshly made 4% paraformaldehyde in 1 Å˜PBS or in 3:1 methanol/acetic acid overnight at 4° C. Eyes were then paraffin embedded with dorsal/ventral orientation and 5 μm sections were collected over 100 μm of retinal depth and processed for hematoxylin/eosin staining. Briefly, retina sections were deparaffinated in xylene and ethanol washed and stained with hematoxylin and eosin Y. Slides were mounted with Permount mounting medium. Over 500 μm of sections/animal were visualized and representative images captured with the Leica DMI6000 microscope. As outer plexiform layer (OPL) collapse was observed in some Nr2e3 treated retinas, the first five layers of cells were counted as inner nuclear layer (INL) and the rest of cell layers were considered as ONL when counting the ONL cell layer number. Quantification of percent observed rescue was determined by comparing treated to control B6 ONL. Cell counts were performed in a double-blinded manner over 100 μm retinal area (N=10/strain/experimental group).

Example 11. Immunohistochemistry: Immunohistochemistry analysis was performed on 10 μm paraffin embedded serial sections from the enucleated mouse eyes. At minimum 100 μm of retina/sample was evaluated by IHC. Briefly, sections were blocked with 2% normal horse serum (#S-2000 VectorLabs, CA) in PBS, and incubated with the following cell type-specific primary antibodies in a 1:200 dilution:rhodopsin (mouse monoclonal, Millipore MAB5356); green/red opsin (rabbit polyclonal, Millipore AB5405); blue opsin (rabbit polyclonal, Millipore AB5407); GFP (1:500, rabbit polyclonal, Abcam ab290). The following day, sections were rinsed with PBS and incubated with the corresponding secondary antibody (1:400 Alexa fluor 488 goat antirabbit, Invitrogen A11008) and nuclei were stained with 4,6-Diamidino-2-Phenylindole, Dihydrochloride (DAPI). Over 500 μm of sections/animal were visualized and representative images of IHC labeling was captured using a Leica DMI6000 fluorescent microscope equipped with the appropriate bandpass filter for each fluorochrome. Cell counts were performed in a double-blinded manner over 100 μm retinal area (N=10/strain/experimental group).

Example 12. Retinal whole mount IHC: Retina whole mounts have been performed as previously described [73]. Microdissection of the retina for whole mounts were performed as follows: the anterior eye segments, including the iris, were removed with a round cut along the limbus using microdissection scissors. The lens was then removed and the retina was gently separated from the pigmented epithelium and the choroidal-scleral complex. Whole retinas were transferred to a 96-well culture plate and immunohistochemistry was performed as follows. Retina cups were blocked with 2% normal horse serum (#S-2000 VectorLabs, CA) in PBS with 0.02% sodium azide, 1% bovine serum albumin (BSA), and 0.1% Triton X 100; and then incubated with the following cell type specific primary antibodies in a 1:100 dilution: rhodopsin (mouse monoclonal, Millipore MAB5356), green opsin (rabbit polyclonal, Millipore AB5405) and blue opsin (rabbit polyclonal, Millipore AB5407) overnight, and incubated with the corresponding secondary antibody (1:200 Alexa fluor, Invitrogen) overnight. Retinas were flowered with radial incisions: two horizontal and two vertical incisions at 3, 6, 9, and 12 clock, starting from the edge toward the optic nerve and cutting ⅔ of the distance from the periphery to the center, giving the retina a crosslike form. Retinas were flat mounted on a microscope slide. Opsin labeling was visualized and images captured using a Leica DMI6000 fluorescent microscope equipped with the appropriate bandpass filter for each fluorochrome (N=10/strain/experimental group).

Example 13. Quantitative real time PCR (qRT-PCR): Total RNA was extracted from whole retinas using the Trizol method. Briefly, 2 μg of total RNA was reverse transcribed using Retroscript (Ambion AM1710) to generate cDNA. The cDNA samples were diluted 1:100 and real time PCR was performed in triplicates for each primer using Sybr Green PCR master mix (Thermo fisher #4309155). The real time PCR primers were designed using NCBI Primer-Blast and were specific for each target gene. Such primers can be found in Supplemental Tables 1 and 2 of Gene Therapy, 2020, published online Mar. 2, 2020 (see doi.org/10.1038/s41434-020-0134-z). Reactions were quantified using an ABI Step One Plus Real Time PCR and analyzed with the corresponding software. Relative expression levels were determined by normalizing cycle threshold values to the amount of (3-actin expressed (1000/2^{Ct} gene—Ct β-actin). Statistical significance of differential expression was assessed using a T-test and P value of <0.05. Results are mean±SEM (N=7). Primers amplifying SV40 from AAV8 vector were used to determine exogenous Nr2e3 expression (Forward primer: AGCAATAGCATC ACAAATTTCACAA (SEQ ID NO:79); Reverse primer: CCAGACATGATAAGATACA TTGA (SEQ ID NO:80)).

Example 14. Chromatin immunoprecipitation RT-PCR: Chromatin immunoprecipitation was performed using P30 C57Bl6/J mouse retinas. A total of 8-10 retinas were used per Chip reaction. Briefly, tissue was dissociated, homogenized, and cross-linked in 37% formaldehyde and sonicated to generate sheared fragments of 400-600 bp. Immunoprecipitation was performed overnight using 1 μg of NR2E3 antibody, goat IgG antibody served as a negative control, and the input (positive control) was not incubated with antibody. Immunoprecipitated samples were reverse cross-linked. Nr2e3 putative target genes were analyzed for nuclear receptor response element (RE) binding site using the classic (AAGTCA (n=1-4) AAGTCA) RE binding sequence of Nr2e3 as determined algorithmically by NUBIscan124. Binding sites were searched for in a maximum of 100 kb upstream region of each gene's start site and into intron 1. Real time primers were selected flanking putative RE sites with an average amplicon size of 200 bp (see Supplementary Table 3 of Gene Therapy, 2020, published online Mar. 2, 2020, doi.org/10.1038/s41434-020-0134-z). Quantitative RT-PCR was performed using 1 μl of 1:100 dilution (input) and 1:10 dilution (samples and immunoglobulin G (IgG) control). All sample data were normalized to IgG control. Results are mean±SEM (N=7/strain/experimental group).

Results

Overexpression of Nr2e3 has no detrimental effect on the retina: C57BL6/J (B6) animals were treated with AAV8-Nr2e3-GFP fusion protein to evaluate any potential detrimental effects of overexpression of Nr2e3, as well as the timing of construct expression post delivery. B6 mice were injected at P0 and evaluated at P7 for 1 month. No observable degeneration was detected in the retina post injection (FIG. 12, Panel (A)). Consistent with clinical findings, AAV delivery of Nr2e3 did not cause aberrant morphological changes, and immunolabeling of rod and cone opsins revealed no observable difference between injected and uninjected animals (FIG. 12, Panel (A)). Functional output of the retina, as detected by electroretinogram (ERG) of rod and cone responses, showed no significant differences between injected and uninjected eyes (FIG. 12, Panel (B)). Examination of AAV8-EGFP-Nr2e3 expression at P7 and P30 revealed that expression of the vector construct was confirmed at P30 (FIG. 12, Panel (C)). These results confirm that overexpression of Nr2e3 by subretinal AAV8-Nr2e3 injection is not detrimental to the retina.

Vector or GFP alone do not affect the retina: Animals were injected with AAV8-EGFP at P0 and evaluated at 1 month to demonstrate that an empty vector alone is not sufficient for or contributes to the rescue observed in Nr2e3 treated animals. Immunohistochemistry analysis confirmed vector expression without any abnormal morphological changes (FIG. 13A). All models except rd7 have 0-1 cells in the ONL by P30, thus GFP is observed in other layers yet has no impact on the disease. Semiquantitative analysis of SV40 polyA gene expression shows no significance difference in expression of SV40 in AAV8-EGFP treated retinas as compared with AAV8-EGFP-Nr2e3 treated animals (FIG. 13B). ERG analysis also suggests that there is neither rescue nor any detrimental effect on functionality of the retina of all mutant strains when treated with empty vector only (AAV8-EGFP) (FIG. 13C).

AAV delivery of Nr2e3 in RP models before disease onset attenuates retinal degeneration: The ability of Nr2e3 to rescue retinal degeneration before disease onset was tested by subretinal delivery of AAV8-Nr2e3 in five mouse models of RP. All models except rd1 were injected at P0 and evaluated at 3-4 months of age. rd1 animals were injected at P0 and evaluated at 1 month of age due to their accelerated rate of disease progression. Although not all models have a clinical phenotype, considerable improvements were observed in the fundus of RhoP23H, rd16, and rd7 mice (FIG. 14). Interestingly, it was observed that the rd16 mice have a red fundus with increased and pronounced vessels (not previously reported). While no vascular leakage has been observed in rd16 mice when examined by fluorescein angiography (data not shown), the fundus observation resolves with Nr2e3 administration. Improvement was observed in the rd7 phenotype, with reduction of retinal spots in AAV8-Nr2e3 treated eyes compared with untreated eyes at 3 months post injection.

Photoreceptor degeneration often disrupts retinal topography and present with abnormal morphology. Histology analysis shows AAV8-Nr2e3 therapy improves retinal morphology and integrity in RP models. The normal mouse retina is comprised of 10-12 layers of rod and cone photoreceptor nuclei in the ONL and 5-6 layers of inner retinal cells in the INL. In the retinal degeneration mouse models evaluated, the INL did not change in nuclei number, and the ONL presented zero or only one layer of cells at the time of evaluation, with the exception of the rd7 model that presents with increased cone cells with whorls and rosettes in the ONL. Hematoxylin/eosin (H/E) staining revealed that subretinal delivery of AAV8-Nr2e3 at P0 rescued photoreceptor cells and helped maintain retinal integrity of RP retinas in all models tested at 1 month (rd1) post treatment, or 3-4 months (Rho−/−, RhoP23H, rd16, and rd7) post treatment (FIG. 15A). The attenuation of disease phenotype, as observed by ONL thickness, varied among each strain. Partial rescue (˜30-80%) of the ONL count in all treated retinas was observed (FIG. 15B). It is also noteworthy that there is no gender bias in the rescue of animals as 50% of the rescued animals are male and 50% are female. Interestingly, retinal whorls and rosettes that are characteristic of the rd7 phenotype, resolved following Nr2e3 treatment, suggesting that the delivery of Nr2e3 at P0 can restore normal retinal development (FIG. 15A). Although a clear boundary between INL, ONL, and OPL was difficult to visualize in some Nr2e3 treated retinas, ONL was significantly increased in the rescued portion of treated retinas (FIG. 15B). rd1 retinas showed a profound rescue of photoreceptor cells, with a total of 6-8 nuclei layers observed in the treated eye. Rho−/−, RhoP23H, and rd16 mice showed a more moderate increase of 3-6 layers of ONL in Nr2e3 treated retinas compared with 0-1 layer in the untreated eyes of each model (FIG. 15C). Although only partial rescue was observed in all models, studies in patients have demonstrated that retention of only a single layer of photoreceptor cells can be enough to maintain minimal visual function suggesting that an increase of even 20% is significant. Thus, Nr2e3 therapy shows great promise in restoring retinal development.

AAV8-Nr2e3 therapy preserves cone and rod opsin expression in five models of RP: Immunohistochemical analysis of blue and green cone opsins and rhodopsin was performed to determine if Nr2e3 therapy can restore opsin expression and thus provide a molecular reset in retinal degeneration models. Eyes from treated and untreated animals were collected at 1 month (rd1) or 3-4 months (Rho−/−, RhoP23H, rd16, and rd7) post injection and labeled with antibodies to green and blue cone opsins and to rhodopsin for rods. Untreated eyes showed no opsin-positive photoreceptor cells, except that of rd7 and Rho−/−. As shown in FIG. 16, rd7 retinas showed an increase in blue opsin and slow progressive loss of all opsins over 5-16 months. These studies show Rho−/− mice have sparse expression of blue and green opsin expressing cones at 1 month (FIG. 16). Interestingly, rhodopsin expression was observed in RhoP23H retinas treated with Nr2e3. The semiquantitative analysis of the blue and green opsin-positive cells shows that there is partial rescue of photoreceptor cells in rd1, Rho−/−, RhoP23H, and rd16 (FIG. 17). En face view of blue and green opsin expression in whole mount retinas of rd1, Rho−/−, RhoP23H, and rd16 Nr2e3 treated animals confirm observations of retinal sections with partial rescue of cone opsin expression in each model, consistent with H/E stain of partial rescue of ONL cells (FIG. 18, opsin expressed regions outlined by a dashed line). By 1-month age, no expression of blue or green opsin was observed in untreated rd1, RhoP23H, and rd16 animals. Similar to IHC in sections, Rho−/− retinas show sparse expression of blue and green opsin. In contrast, all treated animals showed restored blue and green opsin expression (FIG. 18). IHC performed on 1 or 3 month treated animals show consistent expression of the cone opsin genes demonstrating sustained rescue.

Improved ERG responses observed in AAV-Nr2e3 treated RP retinas: RP disease progression results in the loss of rod and cone function that is assessed by abnormal ERG responses. In particular, the visual function of Nr2e3 treated RP retinas was examined in four out of five RP strains, excluding rd7, by recording dark-adapted and light-adapted ERGs to evaluate rod- and cone-driven responses. Consistent with histology and IHC studies, partial rescue is observed in Nr2e3 treated animals compared with untreated animals (FIG. 19A). Each model showed significant percentage increase of the scotopic amplitudes is observed in the treated mutant strains compared with the untreated controls (FIG. 19B).

AAV8-Nr2e3 preserves retinal homeostasis in RP retinas: NHR genes such as Nr2e3 play key roles in modulating homeostasis by regulating many key biological processes and gene networks. Nr2e3 regulates several biological networks in maintaining retina homeostasis in the retina including phototransduction, cell survival, apoptosis, immunity, oxidative stress, ER stress, neuroprotection, and metabolism. Representative subsets of treated animals (rd7, Rho−/−, and rd1) were evaluated for differential expression of genes that function in Nr2e3 regulated pathways. Seventy-five genes were evaluated from eight Nr2e3 modulated biological networks for Nr2e3 RE binding sites. Putative Nr2e3 binding sites in 19 out of 75 genes were identified. Genes with a ≥1.5-fold variance change between the AAV8-Nr2e3 treated and untreated eyes were considered statistically significant. Consistent with the IHC results, the representative strains exhibit a significant change in gene expression of the opsin genes (FIGS. 20A-C). Each strain had a unique set of genes that were differentially expressed between treated and untreated animals. Five out of eight networks were modulated by Nr2e3 treatment in each strain. As expected, the rd7 treated retinas had the greatest number of genes with differential expression in treated vs. untreated retinas (data not shown). In addition, 10 genes were identified by chromatin immunoprecipitation (ChIP)-RTPCR as potential direct targets of Nr2e3, nine of which were differentially expressed in rd7 treated retinas. Interestingly, the ER stress and cell survival factor, inositol—requiring enzyme 1 (Ire1)—is a potential direct target of Nr2e3 and is differentially expressed in all treated animals (data not shown). Considering the unique mutational load of each model, it is not surprising that each mutant had a unique consortia of genes and networks reset by Nr2e3 treatment. Consistent with the finding that Nr2e3 is a dual activator/repressor, these genes were differentially modulated. These results illustrate that mutational load is modulated and balanced by transcription factors, including NHRs, for optimal cellular homeostasis. The upregulation of Ire1 in all treated models suggests at least one common network (ER stress and the promotion of cell survival) through which Nr2e3 modulates and resets homeostasis. Collectively, these findings show that while the specific reset varies among diseases, administration of Nr2e3 to RP diseased retinas restores the homeostatic state of the retina in the presence of disease, thus attenuating disease progression.

While importance of Nr2e3 in photoreceptor development has been well demonstrated, the role of Nr2e3 in the mature retina is less understood. Recent studies reveal a key regulatory role for Nr2e3 in maintaining proper function of mature photoreceptor cells.

The expression of Nr2e3 in all five RP mutant models was evaluated to determine if the loss of Nr2e3 contributes to RP disease. Interestingly, Nr2e3 expression in P7 (rd1) and P30 RP retinas showed a significant decrease of Nr2e3 expression in all RP models except in the rd7−/− model. The present inventors have discovered that the rd7−/− mouse, a functional null of Nr2e3, has high Nr2e3 mRNA expression but lacks protein expression. These results suggest that the loss of Nr2e3 expression likely contributes to the retinal degeneration observed in each model, and the addition of Nr2e3 provides a reset for the for transcriptional signature of treated retinas (FIG. 20). Nr2e3 has been shown to function with other transcription factors such as Nr1d1, neural retinal leucine zipper (Nrl), Cone-rod homeobox (Crx), retinoic acid receptor related orphan receptor alpha (Rora), and thyroid receptor beta (Thrb) to modulate photoreceptor cell fate and retinal function as an activator or suppressor of gene expression. The expression level of five other essential retinal transcription factors (Nr1d1, Nrl, Crx, Rora, and Thrb) were determined in Nr2e3 treated and untreated retinas. Overall, a significant decrease in expression of key retinal transcription factors was reversed following Nr2e3 therapy (FIG. 20). rd1 mice lacked expression of all transcription factors tested except Crx, and these were restored with Nr2e3 therapy, significant down regulation (FIG. 20). Both the rhodopsin models and rd16 exhibited an overall decrease in these transcription factors, and rd7 exhibits an overall reduction of transcription factor expression that is reset following Nr2e3 therapy. See FIG. 20.

AAV8-Nr2e3 rescues retinal degeneration after disease onset: AAV8-Nr2e3 was administered at early to intermediate stage of disease (FIG. 21—Table 1) to determine the efficacy of Nr2e3 modifier gene therapy at a time that better represents clinical presentation. AAV8-Nr2e3 was injected subretinally at P21 and evaluated 2-3 months post injection in Rho−/−, RhoP23H, rd16, and rd7 mice. rd1 mice were injected earlier than P21 as their ONL rapidly degenerates during development. Fundus and histology showed the attenuation of retinal degeneration by Nr2e3 therapy in each model (FIGS. 22A and 22B). As shown previously, improvement varied from ˜30 to 80% of the retina, depending on distribution efficiency throughout the retina. Approximately three to five layers of ONL cells were preserved in Nr2e3 treated animals compared with untreated animals that show less than or equal to one layer of ONL remaining (FIG. 22C). IHC labeling of blue and green cone opsins and rhodopsin further demonstrated the capability of Nr2e3 therapy to rescue photoreceptors after disease onset (FIG. 23). The semiquantitative analysis of the blue and green opsin-positive cells shows a partial rescue of photoreceptor cells in rd1, Rho−/−, RhoP23H, and rd16 (FIG. 24). Rhodopsin rescue is noteworthy in RhoP23H mice when treated at P0 or P21 (FIGS. 16, 23, and 24), emphasizing the unique capability of Nr2e3 to modulate disease mechanism spatially and temporally. To confirm that the rescue observed in Nr2e3 treated retinas is not vector specific, Nr2e3 was packaged in AAV5 and AAV2.7m8. Adult rd7 animals were injected with AAV5-Nr2e3-GFP or AAV2.7m8-Nr2e3 and evaluated clinically before treatment, as well as 1 month post treatment. The presence of GFP in the AAV5-Nr2e3-GFP treated retinas correlated with the region of rescue and corresponded to absence of retinal spots. Reduction of retinal spots and retinal whorls was observed in rd7 animals after 1 month of AAV5 or AAV2.7m8-delivered Nr2e3. OCT images were scanned and captured at the same frame for the same animal before treatment and 1 month post treatment. These results demonstrate that Nr2e3 can ameliorate retinal degeneration outcomes.

Discussion

As clearly demonstrated above, compositions and methods of the invention provide improvement in retinal degeneration using a genetic modifier gene. Composition and methods of the invention clearly show improvement of photoreceptor survival, preservation of retinal structure, a change in gene expression, and stabilization of retinal function. In particular, the cumulative impact toward the restoration of a more homeostatic state of the retina was demonstrated. This restoration allowed for sustained impact and attenuation of RP disease. At least 30-80% improvement at the histological, immunohistochemical, and functional level was observed following treatment with AAV8-Nr2e3. Use of AAV8 is based at least in part on its ability to target specificity toward photoreceptor cells. In addition, Nr2e3 expression was driven by a strong, general promoter rather than a cell type-specific promoter, as they are often not as robust. Experiments disclosed herein show compositions and methods of the invention can be used treat various severity and rate of retinal degeneration disease. Restoration of photoreceptor cells was consistently observed by several methods. Histological analysis revealed regions of rescue in each treated eye. The rescue was demonstrated as an increase in ONL nuclei in specific regions of the retina. Similarly, IHC showed improvement of cone and rod opsin expression in regions of rescue, and it is easy to distinguish regions of rescue in retinal sections and en face views with whole mount retinas.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A recombinant DNA for ameliorating an ocular condition or disease in a subject, said recombinant DNA comprising:
(i) a gene selected from the group consisting of:
(a) a therapeutic gene capable of ameliorating the ocular condition or disease in said subject,
(b) a functional counterpart of a defective gene associated with manifestation of said ocular condition or disease, and
(c) a combination thereof; and
(ii) a delivery vehicle adapted for delivering said gene to cells in an ocular area for treating said ocular condition or disease, wherein said delivery vehicle comprises an adeno-associated virus (AAV) serotype,
wherein said recombinant DNA when transfected to the ocular area of said subject ameliorates said ocular condition or disease in said subject.

2. The recombinant DNA of claim 1, wherein said therapeutic gene is selected from the group consisting of:
(a) human nuclear hormone receptor (hNHR) gene or a fragment thereof, wherein said hNHR gene is selected from the group consisting of NR2E3, NR1C3, NR1D1, RORA, NUPR1, NR2C1, and LXRa;
(b) a growth factor or an angiogenic modulator gene that encodes a peptide selected from the group consisting of:
(i) anti-vegf:
(ii) lens epithelium derived growth factor;
(iii) tumstatin;
(iv) transferrin and tumstatin fusion protein;
(v) fibroblast growth factor;
(vi) platelet-derived growth factor family;
(vii) vascular endothelial growth factor sub-family;
(viii) epidermal growth factor family;
(ix) fibroblast growth factor family;
(x) transforming growth factor-0 superfamily;
(xi) angiopoietin-like family;
(xii) galectins family;
(xiii) integrin superfamily;
(xiv) hepatocyte growth factor;
(xv) angiopoietins;
(xvi) endothelins;
(xvii) hypoxia-inducible factors;
(xviii) insulin-like growth factors;
(xix) cytokines; and
(xx) matrix metalloproteinases gene or a fragment thereof; and
(c) a combination thereof.

3. The recombinant DNA of claim 1, wherein said delivery vehicle comprises adeno-associate virus (AAV) inverted terminal repeat (ITR).

4. The recombinant DNA of claim 1 further comprising (i) a promotor, (ii) an enhancer, (iii) a polyadenylation moiety, (iv) a regulatory switch or (v) a combination thereof.

5. The recombinant DNA of claim 5, wherein said polyadenylation moiety comprises simian virus 40 (SV40) polyadenylation (PolyA) region, bovine growth hormone (bGH) PolyA region, or a combination thereof.

6. The recombinant DNA of claim 1 further comprising cytomegalovirus (CMB) promoter or enhancer, elongation factor 1a (EF1a), chicken (3-actin (CBA) promoter, CAG promotor, a cell/tissue specific promoter, or a combination thereof.

7. A plasmid comprising a recombinant DNA of claim 1.

8. A recombinant adeno-associated virus (rAAV) vector comprising:
(i) a therapeutic gene, wherein said therapeutic gene is selected from the group consisting of:
(a) human nuclear hormone receptor (hNHR) gene or a fragment thereof, wherein said hNHR gene is selected from the group consisting of NR2E3, NR1C3, NR1D1, RORA, NUPR1, NR2C1, and LXRa;
(b) a growth factor or an angiogenic modulator gene that encodes a peptide selected from the group consisting of: (i) anti-vegf: (ii) lens epithelium derived growth factor; (iii) tumstatin; (iv) transferrin and tumstatin fusion protein; (v) fibroblast growth factor; (vi) platelet-derived growth factor family; (vii) vascular endothelial growth factor sub-family; (viii) epidermal growth factor family; (ix) fibroblast growth factor family; (x) transforming growth factor-β superfamily (TGF-β1; activins; follistatin and bone morphogenetic proteins); (xi) angiopoietin-like family; (xii) galectins family; (xiii) integrin superfamily, as well as pigment epithelium derived factor; (xiv) hepatocyte growth factor; (xv) angiopoietins; (xvi) endothelins; (xvii) hypoxia-inducible factors; (xviii) insulin-like growth factors; (xix) cytokines; and (xx) matrix metalloproteinases gene or a fragment thereof; and
(c) a combination thereof;
(ii) at least one functional counterpart of a defective gene associated with manifestation an ocular condition or disease, wherein said ocular condition or disease that is manifested by said defective gene is selected from the group consisting of: (a) Leber congenital amaurosis (“LCA”); (b) retinitis pigmentosa (RP); (c) Cone-rod dystrophy; (d) Macular degeneration; (e) congenital stationary night blindness; (f) synaptic disease; (g) Bardet-Biedl syndrome; (h) Joubert syndrome; (i) Senior-Loken syndrome (CEP290); and (j) Usher syndrome; or
(iii) a combination thereof.

9. The rAAV vector of claim 8 further comprising a naturally occurring adeno-associated virus (AAV) serotype capsid protein.

10. The rAAV vector of claim 8, wherein said hNHR gene is selected from the group consisting of Nr2e3, Nr1d1, Rora, Nupr1, Nr2c1, and LXR.

11. The rAAV vector of claim 8, wherein said NR2E3 gene comprises SEQ ID NO:1 or has at least 90% sequence identity to SEQ ID NO:1.

12. The rAAV vector of claim 8, wherein said NR1D1 gene comprises SEQ ID NO:5 or has at least 90% sequence identity to SEQ ID NO:5.

13. The rAAV vector of claim 8, wherein said RORA gene comprises SEQ ID NO:7 or at least 90% sequence identity to SEQ ID NO:7.

14. The rAAV vector of claim 8, wherein said NR1C3 gene comprises SEQ ID NO:3 or at least 90% sequence identity to SEQ ID NO:3.

15. The rAAV vector of claim 8, wherein said NR2C1 gene comprises SEQ ID NO:11 or has at least 90% sequence identity to SEQ ID NO:11.

16. The rAAV vector of claim 8, wherein said NUPR1 gene comprises SEQ ID NO:9 or has at least 90% sequence identity to SEQ ID NO:9.

17. The rAAV vector of claim 8, wherein said LXRa gene comprises SEQ ID NO:13 or has at least 90% sequence identity to SEQ ID NO:13.

18. The rAAV vector of claim 8 further comprising a capsid protein having SEQ ID NO:71, 72, 73, or 74.

19. A method for treating an ocular condition or ocular disease, said method comprising administering to an ocular tissue of a subject in need of such a treatment a therapeutically effective amount of a composition comprising a recombinant adeno-associated virus (rAAV) vector of claim 8 to treat said subject, wherein said ocular tissue is selected from the group consisting of retinal tissue, choroid tissue, and vitreous tissue.

20. The method of claim 19, wherein said ocular condition or ocular disease comprises Leber congenital amaurosis (LCA), retinitis pigmentosa (RP), enhance S-cone syndrome, Goldmann Favre syndrome, rod-cone dystrophy Bardet-Biedl Syndrome, Achromatopsia, Best Disease (vitelliform macular degeneration), Bardet-Biedl Syndrome, Choroideremia, Macular Degeneration, Stargardt Disease, X-Linked Retinoschisis (XLRS), X-Linked Retinitis Pigmentosa (XLRP), Usher Syndrome, cone-rod dystrophy, Dry-Age related macular degeneration, wet-Age related macular degeneration, or a combination thereof.


SEQUENCE LISTING (80 sequences)

SEQ ID NO: 1 - DNA, 1233 bp, Homo sapiens
atggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcagcggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaacgagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcaaggcccaccaccccagccagcccgtgaggtttgggaaattgctcctgctcctcccgtctttgaggtttatcactgcggaacgcatcgagctcctctttttccgcaagaccatagggaatactccaatggagaagctcctttgtgatatgttcaaaaactag

SEQ ID NO: 2 - protein, 410 aa, Homo sapiens
METRPTALMSSTVAAAAPAAGAASRKESPGRWGLGEDPTGVSPSLQCRVCGDSSSGKHYGIYACNGCSGFFKRSVRRRLIYRCQVGAGMCPVDKAHRNQCQACRLKKCLQAGMNQDAVQNERQPRSTAQVHLDSMESNTESRPESLVAPPAPAGRSPRGPTPMSAARALGHHFMASLITAETCAKLEPEDADENIDVTSNDPEFPSSPYSSSSPCGLDSIHETSARLLFMAVKWAKNLPVFSSLPFRDQVILLEEAWSELFLLGAIQWSLPLDSCPLLAPPEASAAGGAQGRLTLASMETRVLQETISRFRALAVDPTEFACMKALVLFKPETRGLKDPEHVEALQDQSQVMLSQHSKAHHPSQPVRFGKLLLLLPSLRFITAERIELLFFRKTIGNTPMEKLLCDMFKN

SEQ ID NO: 3 - DNA, 1518 bp, Homo sapiens
atgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 4 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 5 - DNA, 1845 bp, Homo sapiens
atgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtga

SEQ ID NO: 6 - protein, 614 aa, Homo sapiens
MTTLDSNNNTGGVITYIGSSGSSPSRTSPESLYSDNSNGSFQSLTQGCPTYFPPSPTGSLTQDPARSFGSIPPSLSDDGSPSSSSSSSSSSSSFYNGSPPGSLQVAMEDSSRVSPSKSTSNITKLNGMVLLCKVCGDVASGFHYGVHACEGCKGFFRRSIQQNIQYKRCLKNENCSIVRINRNRCQQCRFKKCLSVGMSRDAVRFGRIPKREKQRMLAEMQSAMNLANNQLSSQCPLETSPTQHPTPGPMGPSPPPAPVPSPLVGFSQFPQQLTPPRSPSPEPTVEDVISQVARAHREIFTYAHDKLGSSPGNFNANHASGSPPATTPHRWENQGCPPAPNDNNTLAAQRHNEALNGLRQAPSSYPPTWPPGPAHHSCHQSNSNGHRLCPTHVYAAPEGKAPANSPRQGNSKNVLLACPMNMYPHGRSGRTVQEIWEDFSMSFTPAVREVVEFAKHIPGFRDLSQHDQVTLLKAGTFEVLMVRFASLFNVKDQTVMFLSRTTYSLQELGAMGMGDLLSAMFDFSEKLNSLALTEEELGLFTAVVLVSADRSGMENSASVEQLQETLLRALRALVLKNRPLETSRFTKLLLKLPDLRTLNNMHSEKLLSFRVDAQ

SEQ ID NO: 7 - DNA, 1572 bp, Homo sapiens
atggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctaccttaagagccttatgtggacgacatacagaaaagctaatggcatttaaagcaatatacccagacattgtgcgacttcattttcctccattatacaaggagttgttcacttcagaatttgagccagcaatgcaaattgatgggtaa

SEQ ID NO: 8 - protein, 523 aa, Homo sapiens
MESAPAAPDPAASEPGSSGADAAAGSRETPLNQESARKSEPPAPVRRQSYSSTSRGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 9 - DNA, 249 bp, Homo sapiens
atggccaccttcccaccagcaaccagcgccccccagcagcccccaggcccggaggacgaggactccagcctggatgaatctgacctctatagcctggcccattcctacctcggaggtggaggccggaaaggtcgcaccaagagagaagctgctgccaacaccaaccgccccagccctggcgggcacgagaggaaactggtgaccaagctgcagaattcagagaggaagaagcgaggggcacggcgctga

SEQ ID NO: 10 - protein, 82 aa, Homo sapiens
MATFPPATSAPQQPPGPEDEDSSLDESDLYSLAHSYLGGGGRKGRTKREAAANTNRPSPGGHERKLVTKLQNSERKKRGARR

SEQ ID NO: 11 - DNA, 1812 bp, Homo sapiens
atggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagataaaatgtcaacagaaagaagaaaattattgatggagcacatcttcaaactacaggagttttgtaacagcatggttaaactctgcattgatggatacgaatatgcctacctgaaggcaatagtactcttcagtccagatcatccaagcctagaaaacatggaacagatagagaaatttcaggaaaaggcttatgtggaattccaagattatataaccaaaacatatccagatgacacctacaggttatccagactactactcagattgccagctttaagactgatgaatgctaccatcactgaagaattgtttttcaaaggtctcattggcaatatacgaattgacagtgttatcccacatattttgaaaatggagcctgcagattataactctcaaataattggtcacagcatttga

SEQ ID NO: 12 - protein, 603 aa, Homo sapiens
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDKMSTERRKLLMEHIFKLQEFCNSMVKLCIDGYEYAYLKAIVLFSPDHPSLENMEQIEKFQEKAYVEFQDYITKTYPDDTYRLSRLLLRLPALRLMNATITEELFFKGLIGNIRIDSVIPHILKMEPADYNSQIIGHSI

SEQ ID NO: 13 - DNA, 1344 bp, Homo sapiens
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccagggcttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtagagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 14 - protein, 447 aa, Homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRASSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 15 - DNA, 1593 bp, Homo sapiens
atgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagaggaaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtataacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactag

SEQ ID NO: 16 - protein, 530 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEEKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 17 - DNA, 738 bp, Homo sapiens
ccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactga

SEQ ID NO: 18 - protein, 245 aa, Homo sapiens
PGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 19 - DNA, 636 bp, Homo sapiens
atgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaa

SEQ ID NO: 20 - protein, 211 aa, Homo sapiens [sequence omitted]

SEQ ID NO: 21 - DNA, 726 bp, Homo sapiens
atgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctag

SEQ ID NO: 22 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 23 - DNA, 468 bp, Homo sapiens
atggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaa

SEQ ID NO: 24 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 25 - DNA, 11430 bp, Homo sapiens
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcaaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 26 - protein, 2479 aa, Homo sapiens
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 27 - DNA, 1629 bp, Homo sapiens
atgcctctgcgggatgaaaccctccgagaggtgtgggcctctgacagtgggcatgaagaagaaagcctgagcccggaggccccgcggcgccccaaacagcgacccgccccggcacagaggctaaggaagaagaggacggaggcccccgaatccccctgccccacgggatccaagccccggaagcccggagctgggcggacggggaggccgcgggaggagccttccccagacccagcccaggcccgggcgccgcagacggtctacgccaggttcctcagggaccccgaggccaagaagcgcgacccccgggaaacctttctggtagcccgtgccccagacgcggaggacgaggaggaggaggaagaggaggacgaggaggacgaggaagaggaggcagaggaaaagaaagagaaaatccttctgcctcccaagaagcccctgagagagaagagctccgcagacctgaaggagaggagggccaaggcccagggcccaaggggagacctgggaagccctgaccccccaccgaaacctctgcgtgttaggaataaggaagctccagcaggggaggggaccaagatgagaaagaccaagaagaaagggtctggggaggccgacaaggacccctcagggagcccagccagtgcgaggaagagcccagcagccatgtttctggttggggaaggcagtcctgacaagaaagccctgaagaagaaaggcactcccaaaggcgcgaggaaggaggaagaagaggaggaggaggcagctacggtgataaagaagagcaatcaaaagggcaaagccaaaggaaaaggcaaaaagaaagcgaaggaggagagggccccgtctccccccgtggaggtggacgaaccccgggagtttgtgctccggcctgccccccagggccgcacggtgcgctgccggctgacccgggacaaaaagggcatggatcgaggcatgtatccctcctacttcctgcacctggacacggagaagaaggtgttcctcttggctggcaggaaacgaaaacggagcaagacagccaattacctcatctccatcgaccctaccaatctgtcccgaggaggggagaatttcatcgggaagctgaggtccaacctcctggggaaccgcttcacggtctttgacaacgggcagaacccacagcgtgggtacagcactaatgtggcaagccttcggcaggagctggcagctgtgatctatgaaaccaacgtgctgggcttccgtggcccccggcgcatgaccgtcatcattcctggcatgagtgcggagaacgagagggtccccatccggccccgaaatgctagtgacggcctgctggtgcgctggcagaacaagacgctggagagcctcatagaactgcacaacaagccacctgtctggaacgatgacagtggctcctacaccctcaacttccaaggccgggtcacccaggcctcagtcaagaacttccagattgtccacgctgatgaccccgactatatcgtgctgcagttcggccgcgtggcggaggacgccttcaccctagactaccggtacccgctgtgcgccctgcaggccttcgccatcgccctctccagtttcgacgggaagctggcctgcgagtga

SEQ ID NO: 28 - protein, 542 aa, Homo sapiens
MPLRDETLREVWASDSGHEEESLSPEAPRRPKQRPAPAQRLRKKRTEAPESPCPTGSKPRKPGAGRTGRPREEPSPDPAQARAPQTVYARFLRDPEAKKRDPRETFLVARAPDAEDEEEEEEEDEEDEEEEAEEKKEKILLPPKKPLREKSSADLKERRAKAQGPRGDLGSPDPPPKPLRVRNKEAPAGEGTKMRKTKKKGSGEADKDPSGSPASARKSPAAMFLVGEGSPDKKALKKKGTPKGARKEEEEEEEAATVIKKSNQKGKAKGKGKKKAKEERAPSPPVEVDEPREFVLRPAPQGRTVRCRLTRDKKGMDRGMYPSYFLHLDTEKKVFLLAGRKRKRSKTANYLISIDPTNLSRGGENFIGKLRSNLLGNRFTVFDNGQNPQRGYSTNVASLRQELAAVIYETNVLGFRGPRRMTVIIPGMSAENERVPIRPRNASDGLLVRWQNKTLESLIELHNKPPVWNDDSGSYTLNFQGRVTQASVKNFQIVHADDPDYIVLQFGRVAEDAFTLDYRYPLCALQAFAIALSSFDGKLACE

SEQ ID NO: 29 - DNA, 900 bp, Homo sapiens
atgatggcgtatatgaacccggggccccactattctgtcaacgccttggccctaagtggccccagtgtggatctgatgcaccaggctgtgccctacccaagcgcccccaggaagcagcggcgggagcgcaccaccttcacccggagccaactggaggagctggaggcactgtttgccaagacccagtacccagacgtctatgcccgtgaggaggtggctctgaagatcaatctgcctgagtccagggttcaggtttggttcaagaaccggagggctaaatgcaggcagcagcgacagcagcagaaacagcagcagcagcccccagggggccaggccaaggcccggcctgccaagaggaaggcgggcacgtccccaagaccctccacagatgtgtgtccagaccctctgggcatctcagattcctacagtccccctctgcccggcccctcaggctccccaaccacggcagtggccactgtgtccatctggagcccagcctcagagtcccctttgcctgaggcgcagcgggctgggctggtggcctcagggccgtctctgacctccgccccctatgccatgacctacgccccggcctccgctttctgctcttccccctccgcctatgggtctccgagctcctatttcagcggcctagacccctacctttctcccatggtgccccagctagggggcccggctcttagccccctctctggcccctccgtgggaccttccctggcccagtcccccacctccctatcaggccagagctatggcgcctacagccccgtggatagcttggaattcaaggaccccacgggcacctggaaattcacctacaatcccatggaccctctggactacaaggatcagagtgcctggaagtttcagatcttgtag

SEQ ID NO: 30 - protein, 299 aa, Homo sapiens [sequence omitted]

SEQ ID NO: 31 - DNA, 1155 bp, Homo sapiens
atggatgccgctctgctcctgaacgtggaaggggtcaagaaaaccattctgcacgggggcacgggcgagctcccaaacttcatcaccggatcccgagtgatctttcatttccgcaccatgaaatgtgatgaggagcggacagtcattgacgacagtcggcaggtgggccagcccatgcacatcatcatcggaaacatgttcaagctcgaggtctgggagatcctgcttacctccatgcgggtgcacgaggtggccgagttctggtgcgacaccatccacacgggggtctaccccatcctatcccggagcctgaggcagatggcccagggcaaggaccccacagagtggcacgtgcacacgtgcgggctggccaacatgttcgcctaccacacgctgggctacgaggacctggacgagctgcagaaggagcctcagcctctggtctttgtgatcgagctgctgcaggttgatgccccgagtgattaccagagggagacctggaacctgagcaatcatgagaagatgaaggcggtgcccgtcctccacggagagggaaatcggctcttcaagctgggccgctacgaggaggcctcttccaagtaccaggaggccatcatctgcctaaggaacctgcagaccaaggagaagccatgggaggtgcagtggctgaagctggagaagatgatcaatactctgatcctcaactactgccagtgcctgctgaagaaggaggagtactatgaggtgctggagcacaccagtgatattctccggcaccacccaggcatcgtgaaggcctactacgtgcgtgcccgggctcacgcagaggtgtggaatgaggccgaggccaaggcggacctccagaaagtgctggagctggagccgtccatgcagaaggcggtgcgcagggagctgaggctgctggagaaccgcatggcggagaagcaggaggaggagcggctgcgctgccggaacatgctgagccagggtgccacgcagcctcccgcagagccacccacagagccacccgcacagtcatccacagagccacctgcagagccacccacagcaccatctgcagagctgtccgcagggccccctgcagagccagccacagagccacccccgtccccagggcactcgctgcagcactga

SEQ ID NO: 32 - protein, 384 aa, Homo sapiens
MDAALLLNVEGVKKTILHGGTGELPNFITGSRVIFHFRTMKCDEERTVIDDSRQVGQPMHIIIGNMFKLEVWEILLTSMRVHEVAEFWCDTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPAEPPTEPPAQSSTEPPAEPPTAPSAELSAGPPAEPATEPPPSPGHSLQH

SEQ ID NO: 33 - DNA, 828 bp, Homo sapiens
atgaccacagagcaggcaagggggcagcagggcccaaatctggccattggccgtcagaagccccctgcgggggttgtgactcccaagagtgatgcagaggagcccccgttgaccaggaagaggagcaagaaggagagggggctccgagggtctcgaaagcgcactggcagctctggggagcagacaggccccgaggccccggggagcagcaataaccctcccagcactggagaggggccggcgggcgcaccccctgcatcccctgggccggcctcttctcgccagtcccaccgacatcgtcctgactccctgcacgacgctgctcagaggacatacgggcccctgctcaatcgagtcttcgggaaggaccgcgaactgggccccgaggagctagacgagcttcaggccgccttcgaggagtttgacactgaccgtgacggctacatcagccaccgggagctgggtgactgcatgcggaccctgggctacatgcccaccgagatggagctcctggaggtctcgcagcacatcaagatgcgcatgggcggccgtgtggactttgaggagtttgtagaactgataggcccaaagctgagggaggagacggcgcacatgctgggggtgcgagagctgcgcatcgccttccgagagtttgacagggacagggatggacgaattacggtggcggagctgcgggaggcggtaccggctctgctcggggagccgctggcgggtcctgagctggacgagatgctccgagaagtggacctcaatggggatggcaccgtagactttgacgagtttgtgatgatgctctcccgccactga

SEQ ID NO: 34 - protein, 275 aa, Homo sapiens
MTTEQARGQQGPNLAIGRQKPPAGVVTPKSDAEEPPLTRKRSKKERGLRGSRKRTGSSGEQTGPEAPGSSNNPPSTGEGPAGAPPASPGPASSRQSHRHRPDSLHDAAQRTYGPLLNRVFGKDRELGPEELDELQAAFEEFDTDRDGYISHRELGDCMRTLGYMPTEMELLEVSQHIKMRMGGRVDFEEFVELIGPKLREETAHMLGVRELRIAFREFDRDRDGRITVAELREAVPALLGEPLAGPELDEMLREVDLNGDGTVDFDEFVMMLSRH

SEQ ID NO: 35 - DNA, 714 bp, Homo sapiens
atggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctga

SEQ ID NO: 36 - protein, 237 aa, Homo sapiens
MALPPSPLAMEYVNDFDLMKFEVKREPSEGRPGPPTASLGSTPYSSVPPSPTFSEPGMVGATEGTRPGLEELYWLATLQQQLGAGEALGLSPEEAMELLQGQGPVPVDGPHGYYPGSPEETGAQHVQLAERFSDAALVSMSVRELNRQLRGCGRDEALRLKQRRRTLKNRGYAQACRSKRLQQRRGLEAERARLAAQLDALRAEVARLARERDLYKARCDRLTSSGPGSGDPSHLFL

SEQ ID NO: 37 - DNA, 1041 bp, Homo sapiens
atggcgctactgaaagtcaagtttgaccagaagaagcgggtcaagttggcccaagggctctggctcatgaactggttctccgtgttggctggcatcatcatcttcagcctaggactgttcctgaagattgaactccgaaagaggagcgatgtgatgaataattctgagagccattttgtgcccaactcattgatagggatgggggtgctatcctgtgtcttcaactcgctggctgggaagatctgctacgacgccctggacccagccaagtatgccagatggaagccctggctgaagccgtacctggctatctgtgttctcttcaacatcatcctcttccttgtggctctctgctgctttctgcttcggggctcgctggagaacaccctgggccaagggctcaagaacggcatgaagtactaccgggacacagacacccctggcaggtgtttcatgaagaagaccatcgacatgctgcagatcgagttcaaatgctgcggcaacaacggttttcgggactggtttgagattcagtggatcagcaatcgctacctggacttttcctccaaagaagtcaaagatcgaatcaagagcaacgtggatgggcggtacctggtggacggcgtccctttcagctgctgcaatcctagctcgccacggccctgcatccagtatcagatcaccaacaactcagcacactacagttacgaccaccagacggaggagctcaacctgtgggtgcgtggctgcagggctgccctgctgagctactacagcagcctcatgaactccatgggtgtcgtcacgctcctcatttggctcttcgaggtgaccattacaattgggctgcgctacctacagacgtcgctggatggtgtgtccaaccccgaggaatctgagagcgagagccagggctggctgctggagaggagcgtgccggagacctggaaggcctttctggagagtgtgaagaagctgggcaagggcaaccaggtggaagccgagggcgcagacgcaggccaggccccagaggctggctga

SEQ ID NO: 38 - protein, 346 aa, Homo sapiens
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESEGWLLEKSVPETWKAFLESVKKLGKGNQVEAEGAGAGQAPEAG

SEQ ID NO: 39 - DNA, 1047 bp, Homo sapiens
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 40 - protein, 348 aa, Homo sapiens [sequence omitted]

SEQ ID NO: 41 - DNA, 3000 bp, Homo sapiens
atggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtga

SEQ ID NO: 42 - protein, 120 aa, Homo sapiens
MGPAPLPLLLGLFLPALWRRAITEAREEAKPYPLFPGPFPGSLQTDHTPLLSLPHASGYQMGPAPLPLLLGLFLPALWRRAITEAREEAKPYPLFPGPFPGSLQTDHTPLLSLPHASGYQ

SEQ ID NO: 43 - DNA, 1218 bp, Homo sapiens
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctgaggacccagctaaggaaagttatcaggatgcaaatttagtttttgaggagtttgctcgccataatctgaaagatgcaggagaagctgaggaggggaagagagacaagaatgacgttgatgagtga

SEQ ID NO: 44 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 45 - DNA, 6860 bp, Homo sapiens
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcacccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccc

SEQ ID NO: 46 - protein, 2273 aa, Homo sapiens [sequence omitted]

SEQ ID NO: 47 - DNA, 2430 bp, Homo sapiens
atgtttaaatcgctgacaaaagtcaacaaggtgaagcctataggagagaacaatgagaatgaacaaagttctcgtcggaatgaagaaggctctcacccaagtaatcagtctcagcaaaccacagcacaggaagaaaacaaaggtgaagagaaatctctcaaaaccaagtcaactccagtcacgtctgaagagccacacaccaacatacaagacaaactctccaagaaaaattcctctggagatctgaccacaaaccctgaccctcaaaatgcagcagaaccaactggaacagtgccagagcagaaggaaatggaccccgggaaagaaggtccaaacagcccacaaaacaaaccgcctgcagctcctgttataaatgagtatgccgatgcccagctacacaacctggtgaaaagaatgcgtcaaagaacagccctctacaagaaaaagttggtagagggagatctctcctcacccgaagccagcccacaaactgcaaagcccacggctgtaccaccagtaaaagaaagcgatgataagccaacagaacattactacaggctgttgtggttcaaagtcaaaaagatgcctttaacagagtacttaaagcgaattaaacttccaaacagcatagattcatacacagatcgactctatctcctgtggctcttgcttgtcactcttgcctataactggaactgctgttttataccactgcgcctcgtcttcccatatcaaaccgcagacaacatacactactggcttattgcggacatcatatgtgatatcatctacctttatgatatgctatttatccagcccagactccagtttgtaagaggaggagacataatagtggattcaaatgagctaaggaaacactacaggacttctacaaaatttcagttggatgtcgcatcaataataccatttgatatttgctacctcttctttgggtttaatccaatgtttagagcaaataggatgttaaagtacacttcattttttgaatttaatcatcacctagagtctataatggacaaagcatatatctacagagttattcgaacaactggatacttgctgtttattctgcacattaatgcctgtgtttattactgggcttcaaactatgaaggaattggcactactagatgggtgtatgatggggaaggaaacgagtatctgagatgttattattgggcagttcgaactttaattaccattggtggccttccagaaccacaaactttatttgaaattgtttttcaactcttgaattttttttctggagtttttgtgttctccagtttaattggtcagatgagagatgtgattggagcagctacagccaatcagaactacttccgcgcctgcatggatgacaccattgcctacatgaacaattactccattcctaaacttgtgcaaaagcgagttcggacttggtatgaatatacatgggactctcaaagaatgctagatgagtctgatttgcttaagaccctaccaactacggtccagttagccctcgccattgatgtgaacttcagcatcatcagcaaagtcgacttgttcaagggttgtgatacacagatgatttatgacatgttgctaagattgaaatccgttctctatttgcctggtgactttgtctgcaaaaagggagaaattggcaaggaaatgtatatcatcaagcatggagaagtccaagttcttggaggccctgatggtactaaagttctggttactctgaaagctgggtcggtgtttggagaaatcagccttctagcagcaggaggaggaaaccgtcgaactgccaatgtggtggcccacgggtttgccaatcttttaactctagacaaaaagaccctccaagaaattctagtgcattatccagattctgaaaggatcctcatgaagaaagccagagtgcttttaaagcagaaggctaagaccgcagaagcaacccctccaagaaaagatcttgccctcctcttcccaccgaaagaagagacacccaaactgtttaaaactctcctaggaggcacaggaaaagcaagtcttgcaagactactcaaattgaagcgagagcaagcagctcagaagaaagaaaattctgaaggaggagaggaagaaggaaaagaaaatgaagataaacaaaaagaaaatgaagataaacaaaaagaaaatgaagataaaggaaaagaaaatgaagataaagataaaggaagagagccagaagagaagccactggacagacctgaatgtacagcaagtcctattgcagtggaggaagaaccccactcagttagaaggacagttttacccagagggacttctcgtcaatcactcattatcagcatggctccttctgctgagggcggagaagaggttcttactattgaagtcaaagaaaaggctaagcaataa

SEQ ID NO: 48 - protein, 538 aa, Homo sapiens
KSVLYLPGDFVCKKGEIGKEMYIIKHGEVQVLGGPDGTKVLVTLKAGSVFGEISLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQKSVLYLPGDFVCKKGEIGKEMYIIKHGEVQVLGGPDGTKVLVTLKAGSVFGEISLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQ

SEQ ID NO: 49 - DNA, 666 bp, Homo sapiens
atgtcggactctgaggaggagagccaggaccggcaactgaaaatcgtcgtgctgggggacggcgcctccgggaagacctccttaactacgtgttttgctcaagaaacttttgggaaacagtacaaacaaactataggactggatttctttttgagaaggataacattgccaggaaacttgaatgttacccttcaaatttgggatataggagggcagacaataggaggcaaaatgttggataaatatatctatggagcacagggagtcctcttggtatatgatattacaaattatcaaagctttgagaatttagaagattggtatactgtggtgaagaaagtgagcgaggagtcagaaactcagccactggttgccttggtaggcaataaaattgatttggagcatatgcgaacaataaaacctgaaaaacacttacggttttgccaggaaaatggttttagtagccactttgtctcagccaagacaggagactctgtcttcctgtgctttcagaaagttgctgctgaaatccttgggatcaaattaaacaaagcagaaatagaacagtcacagagggtggtgaaggcagatattgtaaactacaaccaggaacctatgtcaaggactgttaaccctcctagaagctctatgtgtgcagttcagtga

SEQ ID NO: 50 - protein, 221 aa, Homo sapiens
MSDSEEESQDRQLKIVVLGDGASGKTSLTTCFAQETFGKQYKQTIGLDFFLRRITLPGNLNVTLQIWDIGGQTIGGKMLDKYIYGAQGVLLVYDITNYQSFENLEDWYTVVKKVSEESETQPLVALVGNKIDLEHMRTIKPEKHLRFCQENGFSSHFVSAKTGDSVFLCFQKVAAEILGIKLNKAEIEQSQRVVKADIVNYNQEPMSRTVNPPRSSMCAVQ

SEQ ID NO: 51 - DNA, 945 bp, Homo sapiens
atggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaa

SEQ ID NO: 52 - protein, 314 aa, Homo sapiens
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD

SEQ ID NO: 53 - DNA, 3459 bp, Homo sapiens
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaacgggccatcaggttccaaaaagttctggaataatgtattaccacattacttggaattgaagtaa

SEQ ID NO: 54 - protein, 1020 aa, Homo sapiens
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVGNDTGQVGPQADTDGEGLQKEVYRHENNNGVDQLDAKEIEKESDGGHSQKESEAEEIDSEKETKLAEIAGMKDLREREKSTKKMSPFFGNLPDRGMNTESEENKDFVKKRESCKQDVIFDSERESVEKPDSYMEGASESQQGIADGFQQPEAIEFSSGEKEDDEVETDQNIRYGRKLIEQGNEKETKPIISKSMAKYDFKCDRLSEIPEEKEGAEDSKGNGIEEQEVEANEENVKVHGGRKEKTEILSDDLTDKAEDHEFSKTEELKLEDVDEEINAENVESKKKTVGDDESVPTGYHSKTEGAERTNDDSSAETIEKKEKANLEERAICEYNENPKGYMLDDADSSSLEILENSETTPSKDMKKTKKIFLFKRVPSINQKIVKNNNEPLPEIKSIGDQIILKSDNKDADQNHMSQNHQNIPPTNTERRSKSCTIL

SEQ ID NO: 55 - DNA, 1053 bp, Homo sapiens
atgggggctggggccagtgctgaggagaagcactccagggagctggaaaagaagctgaaagaggacgctgagaaggatgctcgaaccgtgaagctgctgcttctgggtgccggtgagtccgggaagagcaccatcgtcaagcagatgaagattatccaccaggacgggtactcgctggaagagtgcctcgagtttatcgccatcatctacggcaacacgttgcagtccatcctggccatcgtacgcgccatgaccacactcaacatccagtacggagactctgcacgccaggacgacgcccggaagctgatgcacatggcagacactatcgaggagggcacgatgcccaaggagatgtcggacatcatccagcggctgtggaaggactccggtatccaggcctgttttgagcgcgcctcggagtaccagctcaacgactcggcgggctactacctctccgacctggagcgcctggtaaccccgggctacgtgcccaccgagcaggacgtgctgcgctcgcgagtcaagaccactggcatcatcgagacgcagttctccttcaaggatctcaacttccggatgttcgatgtgggcgggcagcgctcggagcgcaagaagtggatccactgcttcgagggcgtgacctgcatcatcttcatcgcggcgctgagcgcctacgacatggtgctagtggaggacgacgaagtgaaccgcatgcacgagagcctgcacctgttcaacagcatctgcaaccaccgctacttcgccacgacgtccatcgtgctcttccttaacaagaaggacgtcttcttcgagaagatcaagaaggcgcacctcagcatctgtttcccggactacgatggacccaacacctacgaggacgccggcaactacatcaaggtgcagttcctcgagctcaacatgcggcgcgacgtgaaggagatctattcccacatgacgtgcgccaccgacacgcagaacgtcaaatttgtcttcgacgctgtcaccgacatcatcatcaaggagaacctcaaagactgtggcctcttctga

SEQ ID NO: 56 - protein, 350 aa, Homo sapiens
MGAGASAEEKHSRELEKKLKEDAEKDARTVKLLLLGAGESGKSTIVKQMKIIHQDGYSLEECLEFIAIIYGNTLQSILAIVRAMTTLNIQYGDSARQDDARKLMHMADTIEEGTMPKEMSDIIQRLWKDSGIQACFERASEYQLNDSAGYYLSDLERLVTPGYVPTEQDVLRSRVKTTGIIETQFSFKDLNFRMFDVGGQRSERKKWIHCFEGVTCIIFIAALSAYDMVLVEDDEVNRMHESLHLFNSICNHRYFATTSIVLFLNKKDVFFEKIKKAHLSICFPDYDGPNTYEDAGNYIKVQFLELNMRRDVKEIYSHMTCATDTQNVKFVFDAVTDIIIKENLKDCGLF

SEQ ID NO: 57 - DNA, 2583 bp, Homo sapiens
atgggcgaggtgacagcagaggaggtggagaagttcctggactcgaatattggctttgccaaacagtactacaacctccactaccgggccaagctcatctccgacctccttggggccaaggaggctgccgtggacttcagcaactaccactccccgagcagcatggaggagagcgaaatcatctttgatctcctgcgggactttcaggagaatttacagacagagaaatgcatcttcaatgtcatgaagaagctgtgcttcctcctgcaggcagaccgcatgagcctgttcatgtaccggacccgcaatggcatcgcagagctggccaccaggcttttcaatgtccacaaggatgctgtcctcgaggactgcctggtgatgcccgaccaagagatcgtcttccctttggacatgggcatcgtgggccatgtcgcacactctaagaagattgctaacgtccccaacacagaggaggatgagcatttctgtgactttgtggacatcctcacagagtacaagaccaagaacatcttggcttcccccataatgaatgggaaggatgtggtggccataatcatggctgtgaataaagtggatggatcccacttcaccaagagagatgaagagattcttctcaagtacctcaattttgcaaatctaatcatgaaggtgtaccacctgagttacctgcacaactgtgaaactcgacgtggccagatactgctgtggtctgggagcaaagtctttgaagaacttacggacatcgaacgacagttccacaaagccctgtacacagtccgtgctttcctcaactgtgacagatactctgtgggtctcttagacatgaccaagcagaaggaattttttgatgtgtggccggttctgatgggtgaagttccaccttactctggtcccaggactccggatggaagagaaattaacttttacaaggtcattgactacatcctgcatggcaaagaggacatcaaagtcatcccgaatccacctcctgaccattgggctttagtaagcggtctcccagcttatgttgcccagaatggcctgatttgcaacatcatgaatgcgcctgcggaggacttttttgcatttcagaaagaacctctggatgagtctggatggatgattaaaaatgtgctttcaatgccgattgtgaacaagaaggaagaaattgttggagtggccacattttacaatcgtaaagatgggaagccctttgatgaaatggatgagacgctcatggagtctttgactcaatttctgggctggtctgtcttaaatcctgacacctatgagtcaatgaataaacttgaaaataggaaggatattttccaggacatagtaaaatatcatgtgaagtgtgacaatgaagaaattcagaaaatcttgaaaaccagagaggtgtatgggaaggagccatgggagtgtgaggaagaggagctggctgagatcctgcaagcggagctgccagatgcagataaatacgaaattaataaatttcacttcagtgacttacccctaacagaactggagctggtaaaatgtggaatacagatgtattatgagctcaaagtggtggataaatttcacattccacaagaggccctggtgcggttcatgtactccctgagtaagggctaccgcaagatcacctaccacaactggcggcacggcttcaacgtggggcagaccatgttctccctgctggtgacgggaaagctgaagcgctacttcacggacctagaggccttggccatggtcactgctgctttctgccatgacattgaccacagaggcaccaataacctctaccagatgaaatcccagaacccactggccaagctccatgggtcctctatcttggaaagacaccacttggagtttggcaaaacactgctcagagacgagagcctgaatatctttcaaaacctcaatcgtcgacagcatgagcatgccatccacatgatggacattgcaatcattgccacagacctcgccctgtatttcaagaagaggacgatgttccaaaagatcgtggatcagtctaagacatatgagagtgaacaggagtggacacagtacatgatgctggagcagacacggaaggaaatcgttatggccatgatgatgaccgcctgtgatctctcagccatcaccaaaccctgggaggtgcagagccaggtagctctgctggtggctgctgaattctgggaacaaggtgacctggagcgcacggtgctgcaacagaatcccattcccatgatggacagaaacaaagcagatgaactccctaagcttcaagtcggcttcattgactttgtttgcaccttcgtctacaaggaattctcccgtttccacgaggagatcaccccaatgttggacgggatcaccaacaatcgcaaggagtggaaggcgcttgctgatgagtacgatgccaagatgaaggtgcaggaggagaagaagcagaaacagcagtcggccaagtcagcagccgcaggaaatcagccggggggaaaccccagcccagggggtgcaactacatccaagtcctgctgcatccagtaa

SEQ ID NO: 58 - protein, 860 aa, Homo sapiens
MGEVTAEEVEKFLDSNIGFAKQYYNLHYRAKLISDLLGAKEAAVDFSNYHSPSSMEESEIIFDLLRDFQENLQTEKCIFNVMKKLCFLLQADRMSLFMYRTRNGIAELATRLFNVHKDAVLEDCLVMPDQEIVFPLDMGIVGHVAHSKKIANVPNTEEDEHFCDFVDILTEYKTKNILASPIMNGKDVVAIIMAVNKVDGSHFTKRDEEILLKYLNFANLIMKVYHLSYLHNCETRRGQILLWSGSKVFEELTDIERQFHKALYTVRAFLNCDRYSVGLLDMTKQKEFFDVWPVLMGEVPPYSGPRTPDGREINFYKVIDYILHGKEDIKVIPNPPPDHWALVSGLPAYVAQNGLICNIMNAPAEDFFAFQKEPLDESGWMIKNVLSMPIVNKKEEIVGVATFYNRKDGKPFDEMDETLMESLTQFLGWSVLNPDTYESMNKLENRKDIFQDIVKYHVKCDNEEIQKILKTREVYGKEPWECEEEELAEILQAELPDADKYEINKFHFSDLPLTELELVKCGIQMYYELKVVDKFHIPQEALVRFMYSLSKGYRKITYHNWRHGFNVGQTMFSLLVTGKLKRYFTDLEALAMVTAAFCHDIDHRGTNNLYQMKSQNPLAKLHGSSILERHHLEFGKTLLRDESLNIFQNLNRRQHEHAIHMMDIAIIATDLALYFKKRTMFQKIVDQSKTYESEQEWTQYMMLEQTRKEIVMAMMMTACDLSAITKPWEVQSQVALLVAAEFWEQGDLERTVLQQNPIPMMDRNKADELPKLQVGFIDFVCTFVYKEFSRFHEEITPMLDGITNNRKEWKALADEYDAKMKVQEEKKQKQQSAKSAAAGNQPGGNPSPGGATTSKSCCIQ

SEQ ID NO: 59 - DNA, 2565 bp, Homo sapiens
atgagcctcagtgaggagcaggcccggagctttctggaccagaaccccgattttgcccgccagtactttgggaagaaactgagccctgagaatgtggccgcggcctgcgaggacgggtgcccgccggactgcgacagcctccgggacctctgccaggtggaggagagcacggcgctgctggagctggtgcaggatatgcaggagagcatcaacatggagcgcgtggtcttcaaggtcctgcggcgcctctgcaccctcctgcaggccgaccgctgcagcctcttcatgtaccgccagcgcaacggcgtggccgagctggccaccaggcttttcagcgtgcagccggacagcgtcctggaggactgcctggtgccccccgactccgagatcgtcttcccactggacatcggggtcgtgggccacgtggctcagaccaaaaagatggtgaacgtcgaggacgtggccgagtgccctcacttcagctcatttgctgacgagctcactgactacaagacaaagaatatgctggccacacccatcatgaatggcaaagacgtcgtggcggtgatcatggcagtgaacaagctcaacggcccattcttcaccagcgaagacgaagatgtgttcttgaagtacctgaattttgccacgttgtacctgaagatctatcacctgagctacctccacaactgcgagacgcgccgcggccaggtgctgctgtggtcggccaacaaggtgtttgaggagctgacggacatcgagaggcagttccacaaggccttctacacggtgcgggcctacctcaactgcgagcggtactccgtgggcctcctggacatgaccaaggagaaggaattttttgacgtgtggtctgtgctgatgggagagtcccagccgtactcgggcccacgcacgcctgatggccgggaaattgtcttctacaaagtgatcgactacgtcctccacggcaaggaggagatcaaggtcattcccacaccctcagccgatcactgggccctggccagcggccttccaagctacgtggcagaaagcggctttatttgtaacatcatgaatgcttccgctgacgaaatgttcaaatttcaggaaggggccctggacgactccgggtggctcatcaagaatgtgctgtccatgcccatcgtcaacaagaaggaggagattgtgggagtcgccacattttacaacaggaaagacgggaagccctttgacgaacaggacgaggttctcatggagtccctgacacagttcctgggctggtcagtgatgaacaccgacacctacgacaagatgaacaagctggagaaccgcaaggacatcgcacaggacatggtcctttaccacgtgaagtgcgacagggacgagatccagctcatcctgccaaccagagcgcgcctggggaaggagcctgctgactgcgatgaggacgagctgggcgaaatcctgaaggaggagctgccagggcccaccacatttgacatctacgaattccacttctctgacctggagtgcaccgaactggacctggtcaaatgtggcatccagatgtactacgagctgggcgtggtccgaaagttccagatcccccaggaggtcctggtgcggttcctgttctccatcagcaaagggtaccggagaatcacctaccacaactggcgccacggcttcaacgtggcccagacgatgttcacgctgctcatgaccggcaaactgaagagctactacacggacctggaggccttcgccatggtgacagccggcctgtgccatgacatcgaccaccgcggcaccaacaacctgtaccagatgaagtcccagaaccccttggctaagctccacggctcctcgattttggagcggcaccacctggagtttgggaagttcctgctctcggaggagaccctgaacatctaccagaacctgaaccggcggcagcacgagcacgtgatccacctgatggacatcgccatcatcgccacggacctggccctgtacttcaagaagagagcgatgtttcagaagatcgtggatgagtccaagaactaccaggacaagaagagctgggtggagtacctgtccctggagacgacccggaaggagatcgtcatggccatgatgatgacagcctgcgacctgtctgccatcaccaagccctgggaagtccagagcaaggtcgcacttctcgtggctgctgagttctgggagcaaggtgacttggaaaggacagtcttggatcagcagcccattcctatgatggaccggaacaaggcggccgagctccccaagctgcaagtgggcttcatcgacttcgtgtgcacattcgtgtacaaggagttctctcgtttccacgaagagatcctgcccatgttcgaccgactgcagaacaataggaaagagtggaaggcgctggctgatgagtatgaggccaaagtgaaggctctggaggagaaggaggaggaggagagggtggcagccaagaaagtaggcacagaaatttgcaatggcggcccagcacccaagtcttcaacctgctgtatcctgtga

SEQ ID NO: 60 - protein, 854 aa, Homo sapiens
MSLSEEQARSFLDQNPDFARQYFGKKLSPENVAAACEDGCPPDCDSLRDLCQVEESTALLELVQDMQESINMERVVFKVLRRLCTLLQADRCSLFMYRQRNGVAELATRLFSVQPDSVLEDCLVPPDSEIVFPLDIGVVGHVAQTKKMVNVEDVAECPHFSSFADELTDYKTKNMLATPIMNGKDVVAVIMAVNKLNGPFFTSEDEDVFLKYLNFATLYLKIYHLSYLHNCETRRGQVLLWSANKVFEELTDIERQFHKAFYTVRAYLNCERYSVGLLDMTKEKEFFDVWSVLMGESQPYSGPRTPDGREIVFYKVIDYVLHGKEEIKVIPTPSADHWALASGLPSYVAESGFICNIMNASADEMFKFQEGALDDSGWLIKNVLSMPIVNKKEEIVGVATFYNRKDGKPFDEQDEVLMESLTQFLGWSVMNTDTYDKMNKLENRKDIAQDMVLYHVKCDRDEIQLILPTRARLGKEPADCDEDELGEILKEELPGPTTFDIYEFHFSDLECTELDLVKCGIQMYYELGVVRKFQIPQEVLVRFLFSISKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRAMFQKIVDESKNYQDKKSWVEYLSLETTRKEIVMAMMMTACDLSAITKPWEVQSKVALLVAAEFWEQGDLERTVLDQQPIPMMDRNKAAELPKLQVGFIDFVCTFVYKEFSRFHEEILPMFDRLQNNRKEWKALADEYEAKVKALEEKEEEERVAAKKVGTEICNGGPAPKSSTCCIL

SEQ ID NO: 61 - DNA, 2166 bp, Homo sapiens
atgctgctgcctgtgttcaccctgaaactgcgccacaaaatcagcccccgaatggtggccatagggcgctacgacgggactcacccgtgcctggcggccgccacccaaacgggcaaggtttttattcataatcctcatacacggaaccagcatgtcagtgcatccagggtcttccagagccccctggaatctgatgtttctcttctcagcattaaccaggcagtcagctgtctgactgcaggcgtattgaaccctgagcttggctatgatgcccttttagtggggacacagactaatcttttggcttatgatgtctacaataattcggatttgttctacagagaggtagcagatggggcaaatgcaattgtgctggggacattgggagacatttcttcccctcttgcgattattggtggcaattgtgctctgcaaggtttcaatcatgaaggaagtgatctcttttggacggttactggagacaatgttaattccttggccttgtgtgactttgatggtgatggaaagaaagagcttcttgttggatctgaggattttgatatccgagtttttaaggaagatgagattgtggcagaaatgacagaaacagagatagtcacctctctttgtcccatgtatggcagtcgatttggttatgccctttccaatggcacagttggagtttatgacaaaacatcccgatactggagaattaaatcgaaaaatcatgccatgagcattcatgcttttgaccttaattctgatggagtgaatgaactgataactggttggtccaatgggaaggttgatgctcgaagtgaccgaactggggaggtcatctttaaggacaatttttcttctgcaattgccggtgtggtagagggagattaccggatggatggccacatacagttaatctgctgctcagtggatggggaaatccggggctacctgcctggcacggctgagatgaggggcaacctcatggacaccagtgcagagcaggacctgatccgagagctgagtcagaagaagcagaatctgttgctggaactccgtaactatgaggaaaatgccaaggctgaattggccagtccactgaacgaggctgatgggcatcggggcataatcccagccaataccaggctccacaccacgctctcagtcagcctggggaatgagacccaaactgctcatacagaattacgcatttccacttctaatgacaccatcatccgagcagtattgatttttgcagaaggaatttttacaggtgaaagccacgtggtacatcccagcattcacaacctctccagttccatctgcatccctattgtgcctcccaaagatgtccctgtggatctgcacttgaaggcattcgtgggttacagaagcagcacccagtttcatgtatttgaatcgacaagacagctccctcgattctccatgtatgcgctgaccagcctggaccctgccagtgagccaatcagttatgttaactttaccattgcagaacgggcacagagggttgttgtatggctcggtcagaactttctgttaccagaagacactcacattcagaatgctccatttcaagtgtgtttcacatctttacggaatggcggccacctgcatataaaaataaaacttagtggagagatcactataaatactgatgatattgatttggctggtgatatcatccagtcaatggcatcattttttgctattgaagaccttcaagtagaagcggattttcctgtctattttgaggaattacgaaaggtgctagttaaggtggatgaatatcattcagtgcatcagaagctcagtgctgatatggctgatcattctaatttgatccgaagtttgctggtcggagctgaggatgctcgtctgatgagggacatgaaaacaatgaagagtcgttatatggaactctatgaccttaatagagacttgctaaatggatataaaattcgctgtaacaatcacacagagctgttgggaaacctcaaagcagtaaatcaagcaattcaaagagcaggtcgtctgcgggttggaaaaccaaagaaccaggtgatcactgcttgtcgggatgcaattcgaagcaataacatcaacacactgttcaaaatcatgcgagtggggacagcttcttcctag

SEQ ID NO: 62 - protein, 721 aa, Homo sapiens
MLLPVFTLKLRHKISPRMVAIGRYDGTHPCLAAATQTGKVFIHNPHTRNQHVSASRVFQSPLESDVSLLNINQAVSCLTAGVLNPELGYDALLVGTQTNLLAYDVYNNSDLFYREVADGANAIVLGTLGDISSPLAIIGGNCALQGFNHEGSDLFWTVTGDNVNSLALCDFDGDGKKELLVGSEDFDIRVFKEDEIVAEMTETEIVTSLCPMYGSRFGYALSNGTVGVYDKTSRYWRIKSKNHAMSIHAFDLNSDGVNELITGWSNGKVDARSDRTGEVIFKDNFSSAIAGVVEGDYRMDGHIQLICCSVDGEIRGYLPGTAEMRGNLMDTSAEQDLIRELSQKKQNLLLELRNYEENAKAELASPLNEADGHRGIIPANTRLHTTLSVSLGNETQTAHTELRISTSNDTIIRAVLIFAEGIFTGESHVVHPSIHNLSSSICIPIVPPKDVPVDLHLKAFVGYRSSTQFHVFESTRQLPRFSMYALTSLDPASEPISYVNFTIAERAQRVVVWLGQNFLLPEDTHIQNAPFQVCFTSLRNGGHLHIKIKLSGEITINTDDIDLAGDIIQSMASFFAIEDLQVEADFPVYFEELRKVLVKVDEYHSVHQKLSADMADHSNLIRSLLVGAEDARLMRDMKTMKSRYMELYDLNRDLLNGYKIRCNNHTELLGNLKAVNQAIQRAGRLRVGKPKNQVITACRDAIRSNNINTLFKIMRVGTASS

SEQ ID NO: 63 - DNA, 1560 bp, Homo sapiens
atggctgaggagagagtcgcgacgagaactcaatttcctgtatctactgagtctcaaaaaccccggcagaaaaaagctccagagtttcctattttggagaagcagaactggttgattcatcttcattatatccggaaagattatgaagcctgcaaggctgttatcaaagaacagcttcaagagactcagggattgtgtgaatatgctatctatgtccaagcattgatatttcgcctagaaggaaatatccaagaatccctagaactcttccagacatgtgcagttcttagtcctcagagtgctgataacctcaagcaggtggccagatctttatttcttttgggaaaacataaagctgccattgaagtatataatgaagcagctaaactcaaccagaaagattgggagatcagccataacctaggagtttgctacatatacctgaagcagttcaacaaggcacaagaccagttgcacaatgccctgaatcttaataggcacgatctgacttatataatgctggggaagatccacttgctggagggagacttggacaaggccattgaagtctacaagaaagcagtggagttctcaccagaaaatacagagcttcttacaactttaggattactctacttacagctcggcatttaccagaaggcatttgaacatcttggcaatgcactgacttatgaccctaccaactacaaggccatcttggcagcaggcagcatgatgcagacccacggggactttgatgttgccctcaccaaatacagagttgtggcttgtgctgttccagaaagtcctccactctggaataacattggaatgtgtttctttggcaagaagaaatatgtggcggccatcagctgcctgaaacgagccaactacttggcaccctttgattggaagattctgtataatttgggccttgtccatttgaccatgcagcagtatgcatcagcttttcattttctcagtgcggccatcaacttccagccaaagatgggggagctctacatgctcttggcagtggctctgaccaatctggaagatatagaaaatgccaagagagcctacgcagaagcagtccacctggataagtgtaaccctttagtaaacctgaactatgctgtgctgctgtacaaccagggcgagaagaagaacgccctggcccaatatcaggagatggagaagaaagtcagcctactcaaggacaatagctctctggaatttgactctgagatggtggagatggctcagaagttgggagctgctctccaggttggggaggcactggtctggaccaaaccagttaaagatcccaaatcaaagcaccagaccacttcaaccagcaaacctgccagtttccagcagcctctgggctctaatcaagctctaggacaggcaatgtcttcagcagctgcatacaggacgctcccctcaggtgctggaggaacatcccagttcacaaagcccccatctcttcctctggagccagagcctgcggtggaatcaagtccaactgaaacatcagaacaaataagagagaaataa

SEQ ID NO: 64 - protein, 519 aa, Homo sapiens
MAEERVATRTQFPVSTESQKPRQKKAPEFPILEKQNWLIHLHYIRKDYEACKAVIKEQLQETQGLCEYAIYVQALIFRLEGNIQESLELFQTCAVLSPQSADNLKQVARSLFLLGKHKAAIEVYNEAAKLNQKDWEISHNLGVCYIYLKQFNKAQDQLHNALNLNRHDLTYIMLGKIHLLEGDLDKAIEVYKKAVEFSPENTELLTTLGLLYLQLGIYQKAFEHLGNALTYDPTNYKAILAAGSMMQTHGDFDVALTKYRVVACAVPESPPLWNNIGMCFFGKKKYVAAISCLKRANYLAPFDWKILYNLGLVHLTMQQYASAFHFLSAAINFQPKMGELYMLLAVALTNLEDIENAKRAYAEAVHLDKCNPLVNLNYAVLLYNQGEKKNALAQYQEMEKKVSLLKDNSSLEFDSEMVEMAQKLGAALQVGEALVWTKPVKDPKSKHQTTSTSKPASFQQPLGSNQALGQAMSSAAAYRTLPSGAGGTSQFTKPPSLPLEPEPAVESSPTETSEQIREK

SEQ ID NO: 65 - DNA, 6648 bp, Homo sapiens
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 66 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 67 - DNA, 10065 bp, Homo sapiens
atggggcgccatgttgccaccagctgccacgtggcctggcttttggtgctgatctctggatgctggggccaggtgaaccggctgcccttcttcaccaaccacttctttgatacatacctgctgatcagcgaggacacgcctgtgggttcttctgtgacccagttgctggcccaagacatggacaatgaccccctggtgtttggcgtgtctggggaggaggcctctcgcttctttgcagtggagcctgacactggcgtggtgtggctccggcagccactggacagagagaccaagtcagagttcaccgtggagttctctgtcagcgaccaccagggggtgatcacacggaaggtgaacatccaggttggggatgtgaatgacaacgcgcccacatttcacaatcagccctacagcgtccgcatccctgagaatacaccagtggggacgcccatcttcatcgtgaatgccacagaccccgacttgggggcagggggcagcgtcctctactccttccagcccccctcccaattcttcgccattgacagcgcccgcggtatcgtcacagtgatccgggagctggactacgagaccacacaggcctaccagctcacggtcaacgccacagatcaagacaagaccaggcctctgtccaccctggccaacttggccatcatcatcacagatgtccaggacatggaccccatcttcatcaacctgccttacagcaccaacatctacgagcattctcctccgggcacgacggtgcgcatcatcaccgccatagaccaggataaaggacgtccccggggcattggctacaccatcgtttcagggaataccaacagcatctttgccctggactacatcagcggagtgctgaccttgaatggcctgctggaccgggagaaccccctgtacagccatggcttcatcctgactgtgaagggcacggagctgaacgatgaccgcaccccatctgacgctacagtcaccacgaccttcaatatcctggttattgacatcaatgacaatgccccggagttcaacagctccgagtacagcgtggccatcactgagctggcacaggtcggctttgcccttccactcttcatccaggtggtggacaaggatgagaatttgggcctgaacagcatgtttgaggtgtacttggtggggaacaactcccaccacttcatcatctccccgacctccgtccaggggaaggcggacattcgtattcgggtggccatcccactggactacgagaccgtggaccgctacgactttgatctctttgccaatgagagtgtgcctgaccatgtgggctatgccaaggtgaagatcactctcatcaatgaaaatgacaaccggcccatcttcagccagccactgtacaacatcagcctgtacgagaacgtcaccgtggggacctctgtgctgacagtcctggcaactgacaatgatgcaggcacctttggggaagtcagctacttcttcagtgatgaccctgacaggttctcgctggacaaggacacgggactcatcatgctgattgccaggctggactatgagctcatccagcgcttcaccctgacgatcattgcccgggacgggggcggcgaggagaccacaggccgggtcaggatcaatgtgttggatgtcaacgacaacgtgcccaccttccagaaggatgcctacgtgggtgctctgcgggagaacgagccttctgtcacacagctggtgcggctccgggcaacagatgaagactcccctcccaacaaccagatcacctacagcattgtcagtgcatctgcctttggcagctacttcgacatcagcctgtacgagggctatggagtgatcagcgtcagtcgccccctggattatgaacagatatccaatgggctgatttatctgacggtcatggccatggatgctggcaacccccctctcaacagcaccgtccctgtcaccatcgaggtgtttgatgagaatgacaaccctcccaccttcagcaagcccgcctacttcgtctccgtggtggagaacatcatggcaggagccacggtgctgttcctgaatgccacagacctggaccgctcccgggagtacggccaggagtccatcatctactccttggaaggctccacccagtttcggatcaatgcccgctcaggggaaatcaccaccacgtctctgcttgaccgagagaccaagtctgaatacatcctcatcgttcgcgcagtggacgggggtgtgggccacaaccagaaaactggcatcgccaccgtaaacatcaccctcctggacatcaatgacaaccaccccacgtggaaggacgcaccctactacatcaacctggtggagatgacccctccagactctgatgtgaccacggtggtggctgttgacccagacctgggggagaatggcaccctggtgtacagcatccagccacccaacaagttctacagcctcaacagcaccacgggcaagatccgcaccacccacgccatgctggaccgggagaaccccgacccccatgaggccgagctgatgcgcaaaatcgtcgtctctgttactgactgtggcaggccccctctgaaagccaccagcagtgccacagtgtttgtgaacctcttggatctcaatgacaatgaccccacctttcagaacctgccttttgtggccgaggtgcttgaaggcatcccggcgggggtctccatctaccaagtggtggccatcgacctcgatgagggcctgaacggcctggtgtcctaccgcatgccggtgggcatgccccgcatggacttcctcatcaacagcagcagcggcgtggtggtcaccaccaccgagctggaccgcgagcgcatcgcggagtaccagctgcgggtggtggccagtgatgcaggcacgcccaccaagagctccaccagcacgctcaccatccatgtgctggatgtgaacgacgagacgcccaccttcttcccggccgtgtacaatgtgtctgtgtccgaggacgtgccacgcgagttccgggtggtctggctgaactgcacggacaacgacgtgggcctcaatgcagagctcagctacttcatcacaggtggcaacgtggatgggaagttcagcgtgggttaccgcgatgccgttgtgagaaccgtggtgggcctggaccgggagaccacagccgcctacatgctcatcctggaggccatcgacaacggccctgtagggaagcgacacacgggcacagccaccgtgttcgtcactgtcctggatgtgaatgacaaccggcccatctttctgcagagcagctatgaggccagcgtccctgaggacatccctgaaggccacagcatcttgcagctgaaagccacggacgcagatgagggcgagtttgggcgtgtgtggtaccgcatcctccatggtaaccatggcaacaacttccggatccatgtcagcaatgggctcctgatgcgagggccccggcccctggaccgggagcggaactcatcccacgtgctgatagtggaggcctacaaccacgacctgggccccatgcggagctccgtcagggtgattgtgtacgtggaggacatcaacgatgaggcccccgtgttcacacagcagcagtacagccgtctggggcttcgagagaccgcaggcattggaacgtcagtcatcgtggtccaagccacagaccgagactctggggatggtggcctggtgaactaccgcatcctgtcgggcgcagaggggaagtttgagattgacgagagcacagggcttatcatcaccgtgaattacctggactacgagaccaagaccagctacatgatgaatgtgtcggccactgaccaggccccgcccttcaaccagggcttctgcagcgtctacatcactctgctcaacgagctggacgaggccgtgcagttctccaatgcctcatacgaggctgccatcctggagaatctggcactgggtactgagattgtgcgggtccaggcctactccatcgacaacctcaaccaaatcacgtaccgcttcaacgcctacaccagcacccaggccaaagccctcttcaagatagacgccatcacgggtgtgatcacagtccagggcctggtggaccgtgagaagggcgacttctataccttgacagtggtggcagatgacggcggccccaaggtggactccaccgtgaaggtctacatcactgtgctggacgagaatgacaacagcccccggtttgacttcacctccgactcggcggtcagcatacccgaggactgccctgtgggccagcgagtggctactgtcaaggcctgggaccctgatgctggcagcaatgggcaggtggtcttctccctggcctctggcaacatcgcgggggcctttgagatcgtcaccaccaatgactccattggcgaagtgtttgtggccaggcccctggacagagaagagctggatcactacatcctccaggttgtggcttctgaccgaggcacccctccacggaagaaggaccacatcctgcaggtgaccatcctggacatcaatgacaaccctccagtcatcgagagcccctttggatacaatgtcagtgtgaatgagaacgtgggtggaggtactgctgtggtccaggtgagagccactgaccgtgacatcgggatcaacagtgttctgtcctactacatcaccgagggcaacaaggacatggccttccgcatggaccgcatcagcggtgagatcgccacacggcctgccccgcctgaccgcgagcgccagagcttctaccacctggtggccactgtggaggacgagggcaccccaaccctgtcggccaccacgcacgtgtacgtgaccattgtggatgagaatgataacgcgcccatgttccagcagccccactatgaggtgctgctggatgagggcccagacacgctcaacaccagcctcatcaccatccaggcactggacctggatgagggtcccaacggcacagtcacctatgccatcgtcgcaggcaacatcgtcaacaccttccgcatcgacagacacatgggtgtcatcactgctgccaaagagctggactacgagatcagccacggccgctacaccctgatcgtcactgccacagaccagtgccccatcttatcccaccgcctcacctctaccaccacggtgcttgtgaatgtgaatgacatcaacgacaatgtgcctaccttcccccgggactatgagggaccatttgaagtcactgagggccagccggggcccagagtgtggaccttcctggcccatgaccgagactcaggacccaacgggcaggtggagtacagcatcatggatggagaccctctgggggagtttgtgatctctcctgtggagggggtgctaagggtccggaaggacgtggagctggaccgggagaccatcgccttctacaacctgaccatctgtgcccgtgaccgggggatgcccccactcagctccacaatgctggtggggatccgggtgctggacatcaacgacaacgaccctgtgctgctgaacctgcccatgaacatcaccatcagcgagaacagccctgtctccagctttgtcgcccatgtcctggccagtgacgctgacagtggctgcaatgcacgcctcaccttcaacatcactgcgggcaaccgcgagcgggccttcttcatcaatgccacgacagggatcgtcactgtgaaccggcccctggaccgcgagcggatcccagagtacaagctgaccatttctgtgaaggacaacccggagaatccacgcatagccaggagggattatgacttgcttctgatcttcctttctgatgagaatgacaaccaccccctcttcactaaaagcacctaccaggcagaggtgatggaaaactctcccgctggcacccctctcacggtgctcaatgggcccatcctggccctggatgcagaccaagacatctacgccgtggtgacctaccagctgctgggtgcccagagtggcctctttgacatcaacagcagcaccggtgtggtgaccgtgaggtcaggtgtcatcattgaccgggaggcattctcgccacccatcctggagctgctgctgctggctgaggacatcgggctgctcaacagcacggcccacctgctcatcaccatcctggatgacaatgacaaccggcccacctttagccctgccaccctcactgtccatctgctagagaactgcccgcctggattctcagtccttcaagtcacagccacagatgaggacagtggcctcaatggggagctggtctaccgaatagaagctggggctcaggaccgcttcctcattcatctggtcaccggggtcatccgtgttggtaatgccaccatcgacagagaggagcaggagtcctacaggctaacggtggtggccaccgaccggggcaccgttcctctctcgggcacagccattgtcaccattctgatcgatgacatcaatgactcccgccccgagttcctcaaccccatccagacagtgagcgtgctggagtcggctgagccaggcactgtcattgccaatatcacggccattgaccacgacctcaacccaaagctagagtaccacattgtcggcattgtggccaaggacgacactgatcgcctggtgcccaaccaggaggacgcctttgctgtgaatatcaacacaggatctgtaatggtgaagtcccccatgaatcgggagctggttgccacctatgaggtcactctctcagtgattgacaatgccagcgacctaccagagcgctctgtcagtgtgccaaatgccaagctgactgtcaacgtcctggacgtcaatgacaatacgccccagttcaagccctttgggatcacctactacatggagcggatcctggagggggccacccctgggaccacactcattgctgtggcagccgtggaccctgacaagggccttaatgggctggtcacctacaccctgctggacctggtgcccccagggtatgtccagctggaggactcctcggcagggaaggtcattgccaaccggacagtggactacgaggaggtgcactggctcaactttaccgtgagggcctcagacaacgggtccccgccccgggcagctgagatccctgtctacctggaaatcgtggacatcaatgacaacaaccccatctttgaccagccctcctaccaggaggctgtctttgaggatgtgcctgtgggcacaatcatcctgacagtcactgccactgatgctgactcaggcaactttgcactcattgagtacagccttggagatggagagagcaagtttgccatcaaccccaccacgggtgacatctatgtgctgtcttctctggaccgggagaagaaggaccactatatcctgactgccttggccaaagacaaccctggggatgtagccagcaaccgtcgcgaaaattcagtgcaggtggtgatccaagtgctggatgtcaatgactgccggccacagttctccaagccccagttcagcacaagcgtgtatgagaatgagccggcgggcacctcggtcatcaccatgatggccactgaccaggatgaaggtcccaatggagagttgacctactcacttgagggccctggcgtggaggccttccatgtggacatggactcgggcttggtgaccacacagcggccactgcagtcctacgagaagttcagtctgaccgtggtggccacagatggtggagagcccccactctggggcaccaccatgctcctggtggaggtcatcgacgtcaatgacaaccgccctgtctttgtgcgcccacccaacggcaccatcctccacatcagagaggagatcccgctgcgctccaacgtgtacgaggtctacgccacggacaaggatgagggcctcaacggggcggtgcgctacagcttcctgaagactgcgggcaaccgggactgggagttcttcatcatcgacccaatcagcggcctcatccagactgctcagcgcctggaccgcgagtcgcaggcggtgtacagcctcatcttggtggccagcgacctgggccagccagtgccatacgagactatgcagccgctgcaggtggccctggaggacatcgatgacaacgaaccccttttcgtgaggcctccaaaaggcagcccccagtaccagctgctgacagtgcctgagcactcaccacgcggcaccctcgtgggcaacgtgacaggcgcagtggatgcagatgagggccccaacgcgatcgtgtactacttcatcgcagccggcaacgaagagaagaacttccatctgcagcccgatgggtgtctgctggtgctgcgggacctggaccgggagcgagaagccatcttctccttcatcgtcaaggcctccagcaatcgcagctggacacctccccgtggaccctccccaaccctcgacctggttgctgacctcacactgcaggaggtgcgcgttgtgctagaggacatcaacgaccagccaccacgcttcaccaaggctgagtacactgcaggggtggccaccgacgccaaggtgggctcagagttgatccaggtgctggccctggatgcagacattggcaacaacagccttgtcttctacagcattctggccatccactacttccgggcccttgccaacgactctgaagatgtgggccaggtcttcaccatggggagcatggacggcattctgcgcaccttcgacctcttcatggcctacagccccggctacttcgtggtggacattgtggcccgagacctggcaggccacaacgacacggccatcatcggcatctacatcctgagggacgaccagcgcgtcaagatcgtcattaacgagatccccgaccgtgtgcgcggcttcgaggaggagttcatccacctgctctccaacatcactggggccattgtcaatactgacaatgtgcagttccatgtggacaagaagggccgggtgaactttgcgcagacagaactgcttatccacgtggtgaaccgcgataccaaccgcatcctggacgtggaccgggtgatccagatgatcgatgagaacaaggagcagctacggaatcttttccggaactacaacgtcctggacgtgcagcctgccatctctgtccggctgccggatgacatgtctgccctgcagatggcgatcatcgtcctggctatcctcctgttcctggccgccatgctctttgtcctcatgaactggtactacaggactgtacacaagaggaagctcaaggccattgtggctggctcagctgggaatcgtggcttcatcgacatcatggacatgcctaacaccaacaagtactcctttgatggagccaaccctgtgtggctggatcccttctgtcggaacctggagctggccgcccaggcggagcatgaggatgacctaccggagaacctgagtgagatcgccgacctgtggaacagccccacgcgcacccatggaacttttgggcgtgagccagcagctgtcaagcctgatgatgaccgatacctgcgggctgccatccaggagtatgacaacattgccaagctgggccagatcattcgtgaggggccaatcaagggctcgctgctgaaggtggtcctggaggattacctgcggctcaaaaagctctttgcacagcggatggtgcaaaaagcctcctcctgccactcctccatctctgagctgatacagactgagctggacgaggagccaggagaccacagcccagggcagggtagcctgcgcttccgccacaagccaccagtggagctcaaggggcccgatgggatccatgtggtgcacggcagcacgggcacgctgctggccaccgacctcaacagcctgcccgaggaagaccagaagggcctgggccgctcgctggagacgctgaccgctgccgaggccactgccttcgagcgcaacgcccgcacagaatccgccaaatccacacccctgcacaaacttcgcgacgtgatcatggagacccccctggagatcacagagctgtga

SEQ ID NO: 68 - protein, 3354 aa, Homo sapiens
MGRHVATSCHVAWLLVLISGCWGQVNRLPFFTNHFFDTYLLISEDTPVGSSVTQLLAQDMDNDPLVFGVSGEEASRFFAVEPDTGVVWLRQPLDRETKSEFTVEFSVSDHQGVITRKVNIQVGDVNDNAPTFHNQPYSVRIPENTPVGTPIFIVNATDPDLGAGGSVLYSFQPPSQFFAIDSARGIVTVIRELDYETTQAYQLTVNATDQDKTRPLSTLANLAIIITDVQDMDPIFINLPYSTNIYEHSPPGTTVRIITAIDQDKGRPRGIGYTIVSGNTNSIFALDYISGVLTLNGLLDRENPLYSHGFILTVKGTELNDDRTPSDATVTTTFNILVIDINDNAPEFNSSEYSVAITELAQVGFALPLFIQVVDKDENLGLNSMFEVYLVGNNSHHFIISPTSVQGKADIRIRVAIPLDYETVDRYDFDLFANESVPDHVGYAKVKITLINENDNRPIFSQPLYNISLYENVTVGTSVLTVLATDNDAGTFGEVSYFFSDDPDRFSLDKDTGLIMLIARLDYELIQRFTLTIIARDGGGEETTGRVRINVLDVNDNVPTFQKDAYVGALRENEPSVTQLVRLRATDEDSPPNNQITYSIVSASAFGSYFDISLYEGYGVISVSRPLDYEQISNGLIYLTVMAMDAGNPPLNSTVPVTIEVFDENDNPPTFSKPAYFVSVVENIMAGATVLFLNATDLDRSREYGQESIIYSLEGSTQFRINARSGEITTTSLLDRETKSEYILIVRAVDGGVGHNQKTGIATVNITLLDINDNHPTWKDAPYYINLVEMTPPDSDVTTVVAVDPDLGENGTLVYSIQPPNKFYSLNSTTGKIRTTHAMLDRENPDPHEAELMRKIVVSVTDCGRPPLKATSSATVFVNLLDLNDNDPTFQNLPFVAEVLEGIPAGVSIYQVVAIDLDEGLNGLVSYRMPVGMPRMDFLINSSSGVVVTTTELDRERIAEYQLRVVASDAGTPTKSSTSTLTIHVLDVNDETPTFFPAVYNVSVSEDVPREFRVVWLNCTDNDVGLNAELSYFITGGNVDGKFSVGYRDAVVRTVVGLDRETTAAYMLILEAIDNGPVGKRHTGTATVFVTVLDVNDNRPIFLQSSYEASVPEDIPEGHSILQLKATDADEGEFGRVWYRILHGNHGNNFRIHVSNGLLMRGPRPLDRERNSSHVLIVEAYNHDLGPMRSSVRVIVYVEDINDEAPVFTQQQYSRLGLRETAGIGTSVIVVQATDRDSGDGGLVNYRILSGAEGKFEIDESTGLIITVNYLDYETKTSYMMNVSATDQAPPFNQGFCSVYITLLNELDEAVQFSNASYEAAILENLALGTEIVRVQAYSIDNLNQITYRFNAYTSTQAKALFKIDAITGVITVQGLVDREKGDFYTLTVVADDGGPKVDSTVKVYITVLDENDNSPRFDFTSDSAVSIPEDCPVGQRVATVKAWDPDAGSNGQVVFSLASGNIAGAFEIVTTNDSIGEVFVARPLDREELDHYILQVVASDRGTPPRKKDHILQVTILDINDNPPVIESPFGYNVSVNENVGGGTAVVQVRATDRDIGINSVLSYYITEGNKDMAFRMDRISGEIATRPAPPDRERQSFYHLVATVEDEGTPTLSATTHVYVTIVDENDNAPMFQQPHYEVLLDEGPDTLNTSLITIQALDLDEGPNGTVTYAIVAGNIVNTFRIDRHMGVITAAKELDYEISHGRYTLIVTATDQCPILSHRLTSTTTVLVNVNDINDNVPTFPRDYEGPFEVTEGQPGPRVWTFLAHDRDSGPNGQVEYSIMDGDPLGEFVISPVEGVLRVRKDVELDRETIAFYNLTICARDRGMPPLSSTMLVGIRVLDINDNDPVLLNLPMNITISENSPVSSFVAHVLASDADSGCNARLTFNITAGNRERAFFINATTGIVTVNRPLDRERIPEYKLTISVKDNPENPRIARRDYDLLLIFLSDENDNHPLFTKSTYQAEVMENSPAGTPLTVLNGPILALDADQDIYAVVTYQLLGAQSGLFDINSSTGVVTVRSGVIIDREAFSPPILELLLLAEDIGLLNSTAHLLITILDDNDNRPTFSPATLTVHLLENCPPGFSVLQVTATDEDSGLNGELVYRIEAGAQDRFLIHLVTGVIRVGNATIDREEQESYRLTVVATDRGTVPLSGTAIVTILIDDINDSRPEFLNPIQTVSVLESAEPGTVIANITAIDHDLNPKLEYHIVGIVAKDDTDRLVPNQEDAFAVNINTGSVMVKSPMNRELVATYEVTLSVIDNASDLPERSVSVPNAKLTVNVLDVNDNTPQFKPFGITYYMERILEGATPGTTLIAVAAVDPDKGLNGLVTYTLLDLVPPGYVQLEDSSAGKVIANRTVDYEEVHWLNFTVRASDNGSPPRAAEIPVYLEIVDINDNNPIFDQPSYQEAVFEDVPVGTIILTVTATDADSGNFALIEYSLGDGESKFAINPTTGDIYVLSSLDREKKDHYILTALAKDNPGDVASNRRENSVQVVIQVLDVNDCRPQFSKPQFSTSVYENEPAGTSVITMMATDQDEGPNGELTYSLEGPGVEAFHVDMDSGLVTTQRPLQSYEKFSLTVVATDGGEPPLWGTTMLLVEVIDVNDNRPVFVRPPNGTILHIREEIPLRSNVYEVYATDKDEGLNGAVRYSFLKTAGNRDWEFFIIDPISGLIQTAQRLDRESQAVYSLILVASDLGQPVPYETMQPLQVALEDIDDNEPLFVRPPKGSPQYQLLTVPEHSPRGTLVGNVTGAVDADEGPNAIVYYFIAAGNEEKNFHLQPDGCLLVLRDLDREREAIFSFIVKASSNRSWTPPRGPSPTLDLVADLTLQEVRVVLEDINDQPPRFTKAEYTAGVATDAKVGSELIQVLALDADIGNNSLVFYSILAIHYFRALANDSEDVGQVFTMGSMDGILRTFDLFMAYSPGYFVVDIVARDLAGHNDTAIIGIYILRDDQRVKIVINEIPDRVRGFEEEFIHLLSNITGAIVNTDNVQFHVDKKGRVNFAQTELLIHVVNRDTNRILDVDRVIQMIDENKEQLRNLFRNYNVLDVQPAISVRLPDDMSALQMAIIVLAILLFLAAMLFVLMNWYYRTVHKRKLKAIVAGSAGNRGFIDIMDMPNTNKYSFDGANPVWLDPFCRNLELAAQAEHEDDLPENLSEIADLWNSPTRTHGTFGREPAAVKPDDDRYLRAAIQEYDNIAKLGQIIREGPIKGSLLKVVLEDYLRLKKLFAQRMVQKASSCHSSISELIQTELDEEPGDHSPGQGSLRFRHKPPVELKGPDGIHVVHGSTGTLLATDLNSLPEEDQKGLGRSLETLTAAEATAFERNARTESAKSTPLHKLRDVIMETPLEITEL

SEQ ID NO: 69 - DNA, 699 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactga

SEQ ID NO: 70 - protein, 232 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 71 - DNA, 2211 bp, Adeno-associated virus-1
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 72 - DNA, 2208 bp, adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 73 - DNA, 2175 bp, Adeno-associated virus 5
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 74 - DNA, 2217 bp, Adeno-associated virus 8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 75 - DNA, 33 bp, Artificial Sequence
  Forward Primer for mRNA of a B6 mouse retina
gctgtacaagggcggatgagctctacagtggct

SEQ ID NO: 76 - DNA, 26 bp, Artificial Sequence
  Reverse Primer for mRNA of a B6 mouse retina
ataccggttggcactcccaactagtt

SEQ ID NO: 77 - DNA, 33 bp, Artificial Sequence
  FORWARD PRIMER IN EXAMPLE 6
cctaagcttatgagctctacagtggctgcctcc

SEQ ID NO: 78 - DNA, 45 bp, Artificial Sequence
  REVERSE PRIMER IN EXAMPLE 6
atcgaattcggatccggtaccctagtttttgaacatgtcacacag

SEQ ID NO: 79 - DNA, 25 bp, Artificial Sequence
  SV40 amplyfing Forward Primer
agcaatagcatcacaaatttcacaa

SEQ ID NO: 80 - DNA, 23 bp, Artificial Sequence
  SV40 amplifying reverse primer
ccagacatgataagatacattga